(19) United States
(12) Patent Application Publication
(10) Pub. No.: US 2007/0004035 A1
(43) Pub. Date: Jan. 4, 2007

(54) Gene therapy methods and cell growth and cell transplant devices for use therein

(76) Inventor: James Sitzmann, Zionsville, IN (US)

Correspondence Address: DAVID G. HENRY, 900 Washington Ave., P.O. Box 1470, Waco, TX, 76703-1470, US

(21) Appl. No.: 11/171,637

(22) Filed: Jun. 30, 2005

(51) Int. Cl. C12N 9/00; C12N 5/08; C12N 5/06

(52) U.S. Cl. 435/325; 435/366; 435/183; 435/289.1

(57) ABSTRACT

An artificial tendon, ligament, bone and skin panel are provided, made of biocompatible materials and capable of being seeded and eventually coated with selected cell types. Seeded or coated artificial tendon, ligament, bone or skin panels can be shaped and implanted in mammals. Implantable cell growth cages may be seeded with selected cell types and used to produce desired gene expression products within a mammal. Gene expression products may be harvested from transfected animal cells cultured in a cell growth chamber.

### FIELD OF THE INVENTION

The present invention relates generally to the field of biocompatible, implantable replacement body components. In particular, the invention relates to artificial tendons, bones, and skin, methods of making the same, and artificial bodily components made therefrom. The present invention further relates to the field of biocompatible, implantable cell growth chambers useful in facilitating in situ transplanted cell growth. The present invention further relates to equipment useful in cell culture operations.

### BACKGROUND OF THE INVENTION

Mammalian organisms, including humans, are occasionally born with various body components missing, non-functional, or dysfunctional. Further, throughout life, organisms suffer disease and injury to their bodies. Some defects or damage can be accommodated naturally. Other problems with body components may be treated with a variety of medical techniques. Modern medicine (both human and veterinary) is continuously advancing new treatments for the problems faced by humans and other mammalian organisms.

One example of damage is a stretched, torn, or otherwise damaged tendon. This commonly occurs to humans when engaged in athletic activity and causes pain and reduced mobility. It is also a problem of some economic significance in the race horse industry. For many decades, efforts have focused on creating a lasting solution to irreparably damaged tendons. No approach has yet proven to be completely successful in replicating the natural action of the native tendon while being well tolerated by the recipient.

Further examples are the ever-present problems of broken or misshapen bones. Some solutions to bone repair are available. For relatively straight breaks with little associated tissue trauma, merely resetting the bone and immobilizing it for a time sufficient for repair to naturally occur may be an acceptable treatment. Currently there are devastating diseases and injuries that require entire bone replacements. In addition, with the increasing longevity of the population, more individuals will live to an age to be affected by diseases such as osteoporosis, making severely broken bones more common and a need to provide replacement bones more urgent. Preferably, these new treatments should be able to mimic the natural shape, weight, and strength of natural bone while being immunologically acceptable to the patient.

Medicine is also advancing to provide the replacement or reconstruction of damaged external organs. Burn victims are often treated with multiple skin grafts and occasionally also need replacement or reconstructed noses and ears. Other accidental or disease related conditions cause patients to damage a region of their body surface in a way that requires a cosmetically reasonable, medically sound solution. Moreover, people desire a normal, or sometimes even an improved, appearance for themselves and for their pets. It would be beneficial for practitioners to have additional tools and methods to meet this continuously growing demand, and to meet the demand in ways that are more medically beneficial and safer for the patient and at the same time relatively easily applied by the practitioner.

Further challenges to medical professionals abound. Certain patients are unable to produce materials necessary for normal body function. Also, it may be desirable to enhance the production of materials beyond normal levels or in patients who would not normally produce those materials. Examples include supplemental insulin production in diabetics, or additional hormonal production for patients suffering from abnormally low levels. Also, since hormone replacement therapy has become a commonly prescribed treatment for post-menopausal women, alternatives to drug therapy, which can have side effects in addition to cost, should be made available. A method that could help achieve the goal of normal material production in an efficient and biocompatible matter would be welcome in the art.

There also are known treatments using compounds produced through transfection of an animal or microbe with a desired gene. Current culturing methods are not always efficient at culturing the desired cells while extracting the desired product. An improved device capable of maintaining cell culture while facilitating removal of desired cell-produced products would be an advance in the art and make treatments more available and affordable.

As noted above in discussions regarding particular fields of research, a common problem associated with modern medical treatment is an unintended or undesirable immune response. As naturally occurring or naturally produced materials are increasingly used in treatments, methods that reduce or eliminate the immunological complications associated with treatment will continue to be sought. For example, the current practice of allowing a patient to have blood removed and stored prior to a surgical procedure allows a surgeon to transfuse the patient with their own blood. This eliminates concern over blood type matching and disease transmission due to non-self transfusion. More advanced self treatments that offer similar advantages to the patient and practitioner are needed in the medical arts.

### SUMMARY OF THE INVENTION

In certain embodiments, the invention provides biocompatible replacement body components.

In other embodiments, the invention provides devices useful in maintaining in vivo cell cultures to produce desired materials.

In another embodiment, the invention provides an improved device for facilitating growth of transfected cells and extraction of the products produced thereby.

In still another embodiment, the invention provides treatments and devices that are recognized by a patient's immunological system as natural, self-produced materials thus eliminating any significant immunological response to the treatment or device.

According to a first embodiment of the present invention, an artificial tendon or ligament is provided, comprising a plurality of elongated fibers each having a first end and a second end, a first end fiber at the first end of the elongated fibers; and a second end fiber at the second end of the elongated fibers, wherein the first and the second end fibers connect the respective first and second ends of the elongated fibers. Optionally, the artificial tendon or ligament can further comprise at least one cross-connecting fiber attached to the elongated fibers between the first ends and the second ends, or it can further comprise a layer of fibroblast or tendon cells on the elongated fibers.

According to a further embodiment of the present invention, an artificial weight-bearing bone is provided, comprising a plurality of elongated members, a plurality of circular fibers and a layer of bone or cartilage cells on at least one of the elongated members, wherein the elongated members form a generally cylindrical structure, the circular fibers are located around and permanently attached to the elongated members, and the circular fibers are spaced along the length of the elongated members. Optionally, the elongated members may be comprised of metal. Such an artificial weight-bearing bone may be, for example, a femur, tibia, fibula, rib, clavicle, humerus, radius or ulna. Optionally, the artificial weight-bearing bone may mimic the shape of a naturally occurring bone or portion thereof.

According to a further embodiment of the present invention, an artificial weight-bearing bone is provided, comprising a plurality of elongated members, a plurality of circular fibers, and at least one layer of bone or cartilage cells on at least one of the elongated members, wherein the elongated members form a structure that mimics the shape of a natural bone or portion thereof, and wherein each of the circular fibers are located around and permanently attached to at least two of the elongated members. Optionally, the elongated members may be comprised of metal. Such an artificial weight-bearing bone may be, for example, a scapula, vertebra, inferior maxillary, sternum, patella or os innominatum.

According to a further embodiment of the present invention, an artificial non weight-bearing bone is provided, comprising at least one artificial bone scaffold and at least one layer of bone or cartilage cells on the artificial bone scaffold, wherein the artificial bone scaffold is configured to mimic the shape of a natural bone or a portion thereof. Optionally, the artificial bone may further comprise a layer of cell growth matrix located along the inner or outer surface of the artificial bone scaffold. For example, the artificial non weight-bearing bone may be an artificial nose or an artificial ear.

According to a further embodiment of the present invention, an artificial skin panel is provided, comprising a fibrous matrix and at least one cellular coating on the fibrous matrix, wherein the at least one cellular coating is comprised of cells selected from the group consisting of superficial cutaneous cells and deep cutaneous cells.

According to a further embodiment of the present invention, an artificial cell growth cage is provided, comprising a fibrous matrix and a plurality of cells on and within the fibrous matrix, wherein the cells are capable of producing a desired gene expression product. Optionally, the artificial cell growth cage may be tube shaped and may have an outer diameter of approximately 10-16 gauge and an inner diameter of approximately 0.5-0.05 mm. An alternative option is to have a disk-shaped artificial cell growth cage which may have an outer diameter of approximately 1-3 cm, a length of approximately 3-6 cm and a width of approximately 25-100 mm.

According to a further embodiment of the present invention, a cellular growth chamber is provided, comprising a vessel, an opening in the vessel that allows insertion and removal of a plurality of porous inner tubes and that is sealingly closeable and a sealable port providing a opening in the vessel and allowing inlet and outlet of cell culture solution.

According to a further embodiment of the present invention, a method of obtaining a substance is provided, comprising culturing cells in a cellular growth chamber according to the previous embodiment and isolating a substance produced by the cells. Optionally, the isolated substance may be myostatin or follistatin.

Other objects, advantages and novel features of the present invention will become apparent from the following detailed description and accompanying drawings.

### BRIEF DESCRIPTION OF THE DRAWINGS

FIG. 1A shows a first embodiment of a tendon or ligament scaffold;

FIG. 1B shows a second embodiment of a tendon or ligament scaffold;

FIG. 1C shows a third embodiment of a tendon or ligament scaffold;

FIG. 2 a perspective view of a weight bearing bone scaffold;

FIG. 3 is a perspective view of a cell growth matrix for use with the scaffold shown in FIG. 2;

FIG. 4A is a side view of a weight bearing bone scaffold with a cell growth matrix;

FIG. 4B shows a cross section from region I-I of the weight bearing bone scaffold of FIG. 4A;

FIG. 5A shows a first embodiment of a non-weight bearing bone scaffold;

FIG. 5B shows a variation in shape of a first embodiment of a non-weight bearing bone scaffold;

FIG. 6A shows a second embodiment of a non-weight bearing bone scaffold;

FIG. 6B shows a variation in shape of a second embodiment of a non-weight bearing bone scaffold;

FIG. 6C shows a further variation in shape of a second embodiment of a non-weight bearing bone scaffold;

FIG. 7A is a schematic drawing of an assembly of non-weight bearing bone scaffolds;

FIG. 7B is a schematic drawing of an assembly of non-weight bearing bone scaffolds;

FIG. 8A is a front view of an artificial ear;

FIG. 8B is a side view of an artificial ear;

FIG. 9 is a schematic drawing of a configuration of artificial skin scaffold panels;

FIG. 10 is a schematic drawing of a further configuration of artificial skin scaffold panels;

FIG. 11 depicts a tube shaped cell growth cage;

FIG. 12 is a cross-sectional view of the tube shaped cell growth cage of FIG. 11;

FIG. 13 depicts a disk shaped cell growth cage;

FIG. 14 shows a coiled disk shaped cell growth cage ready for insertion into a capsule;

FIG. 15A is a cut away side view of a cell growth chamber;

FIG. 15B is a cross sectional view of the cell growth chamber of FIG. 15A taken along line II—II; and

FIG. 16 is a side view of an inner tube of a cell growth chamber.

### DETAILED DESCRIPTION OF PREFERRED EMBODIMENTS

The present invention may be understood more readily by reference to the following detailed description of particular embodiments of the invention and the specific examples. The terminology used herein is for the purpose of describing particular embodiments only and is not intended to be limiting.

### EXAMPLE I

Fabrication of an Artificial Tendon or Ligament Matrix

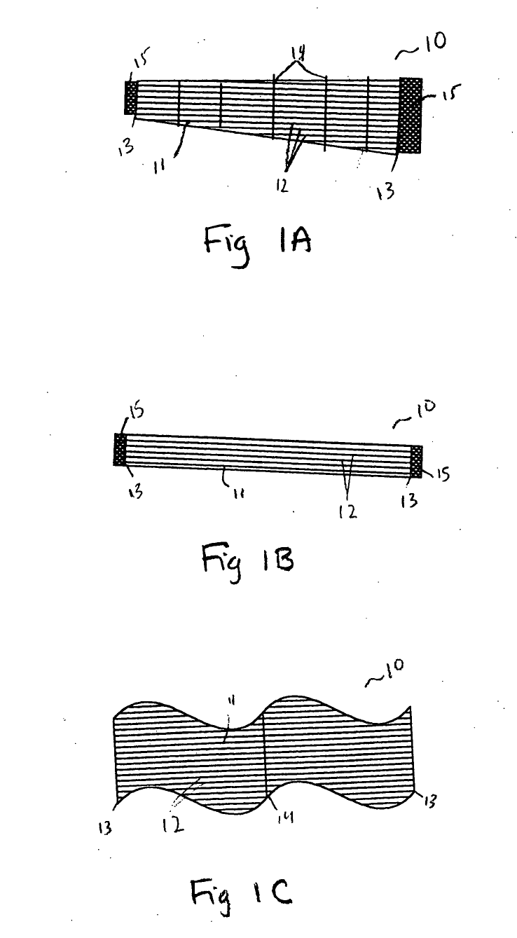

A matrix is prepared with shape and size dependent on the intended use for a replacement tendon. Tendons that will be used to attach major bone and muscles together and bear significant mechanical stress and weight are preferably long and broad. FIG. 1A shows the matrix for an artificial tendon or ligament. The tendon 10 may be used where there is a need for the tendon to bear weight. The body 11 of artificial tendon 10 can be trapezoidal in shape, as shown. Body 11 is comprised of longitudinal fibers 12. Longitudinal fibers 12 are secured at their first and second ends with end fibers 13. Because the intended use of the artificial tendon shown in FIG. 1A includes major stresses, multiple cross-connecting fibers 14 are provided within body 11 to add strength and durability. The configuration of two sets of parallel fibers arranged perpendicular to one another, as shown, is a preferred embodiment. Alternate embodiments include cross-connecting fibers at angles other than 180°, using more than two sets of parallel fibers, and using fiber sets that are not entirely parallel. To improve the durability and functionality of artificial tendon 10, a configuration of fibers that mimics the dense fibrous nature of a natural tendon may be most preferred. Connecting strips 15 may be provided on artificial tendon 10 to facilitate suturing or bonding of artificial tendon 10 to the appropriate muscle or bone. While connecting strips 15 are depicted as rectangular strips placed or interwoven along the opposite end regions of body 11, they may exist in a variety of shapes, including strips, blocks, loops, hooks, etc.

A variety of materials may be used for the fibers, it may be preferred to use a variety of different materials for each artificial tendon or ligament. The fibers should possess a desired tensile strength to resist rupture, fracture, or breakage due to stresses. Stresses to be considered include muscular, weight, gravity, application of force, or movement. Fibers that would retain their shape and function with use are desired. A preferred quality of the fiber is that it will initiate or cause inflammatory reaction in the host tissue. The selected fiber should preferably also allow cell growth, possibly even causing cell growth, such as fibroblast or scar tissue cells.

Examples of materials include, but are not limited to, latex, rubber, elastic, glass, ceramic, plastic, and polyester. Materials also include silastic, proline, methacrylate, nylon, dacron, polydimethylsiloxane, polyurethane, polyethylene, vicryl, and goretex. Further materials include polypropylene, octyl-2-cyanoacrylate, polymethylacrylate, polylactide, aramid, polystyrene, poly-L-lysine, terephthalate (PET), neutral polymer, silica polymer, polygalctide, hydroxylapatite, latex, rubber, elastic, glass, ceramic, plastic or polyester. Other suitable materials may also be used.

Fiber sizes may be characterized as either small, medium, or large. Small fibers range in size from 0.1 μm to 2.0 μm, including diameters of 0.1 μm, 0.2 μm, 0.3 μm, 0.4 μm, 0.5 μm, 0.6 μm, 0.8 μm, 1.0 μm, 1.2 μm, 1.5 μm, 1.8 μm, and 2.0 μm. Medium fibers have a diameter of 3.0 μm to 50 μm, including 3.0 μm, 4.0 μm, 5.0 μm, 6.0 μm, 8.0 μm, 10.0 μm., 15.0 μm, 20 μm, 30 μm, 40 μm, and 50 μm. Large fibers have diameters ranging from 100 μm to 1,000 μm, including sizes such as 150 μm, 200 μm, 250 μm, 300 μm, 350 μm, 400 μm, 500 μm, 600 μm, 800 μm, and 1,000 μm. Although certain sizes are disclosed, any size within or approximating the listed ranges may be used.

To hold the fibers together, they are bonded with other fibers, or using glue or a weaving technique. For certain fiber materials, a thermal bond may be created with the application of heat to melt fibers together at a specific point. Chemical adhesives may also be used, such as chemical adhesive glues. Fibers, plastic, or silastic attachments may also be employed. The fibers may be woven in a knot-lock style of weaving-knitting to secure the fibers together. A variety and a combination of the attachment techniques may be employed.

The fibers may be attached to the appropriate bone or muscle location with a suture, which may be absorbable or non-absorbable. They may also be attached with metal or plastic screws. Other appropriate attachment devices as known in the art may be employed.

Artificial tendons or ligaments can be prepared to serve all the uses of natural tendons or ligaments. Tendons in the hand, wrist, and foot are typically long and narrow, but they may be damaged by disease, injury, or may be misshapen or missing due to some defect. In non-human mammals, similarly defective tendons exist and could be replaced by artificial tendons according to the present invention. As shown in FIG. 1B, an artificial tendon 10 suitable for use in hands, wrists or feet would preferably have a long, narrowly shaped body 11. Sufficient longitudinal fibers 12 are provided to give shape and stability to artificial tendon 10, in this embodiment no cross-connecting fibers are shown. This is because of the relatively low mechanical demands on tendons in some regions. Optional connecting strips 15 are also shown.

Just as natural tendons have a variety of shapes, sizes and purposes, so do the artificial tendons of the present invention. A further embodiment is shown in FIG. 1C, depicting a free form shape. Body 11 has a wave-like shape that may be desirable in certain applications. Longitudinal fibers 12 and a cross-connecting fiber 14 are provided. The free form nature of this embodiment allows for creation of a matrix that will fit any application, including applications where, due to reconstructive work or other abnormal situation, the tendon must be configured in a non-natural shape.

The matrix may be prepared by cutting or shaping existing suitable mesh framework, or by creating an interwoven or otherwise interconnected series of fibers. For example, #10 nylon fibers may be prepared in a desired shape and secured together with sealant such as silicone, polyurethane, or polyethylene. The mesh-type nature of the matrix preferably has a pore size of no less than 0.1 μm and no greater than 1,000 μm. Typical sizes for artificial tendons according to the present invention include lengths from 2 cm to 10 cm and widths of from 0.5 cm to 2 cm.

The artificial tendon or ligament matrix may be directly implantable without any further modification. Optionally, chemicals or materials that facilitate cell migration and growth may be applied to the matrix. A further option is to coat the artificial tendon or ligament with cells prior to implant, as described below in Example II.

### EXAMPLE II

Deposit of Cellular Coating on an Artificial Tendon

Prior to implanting an artificial tendon as described above, tendon or fibroblast cells may be coated on to the bioartificial matrix that comprises the artificial tendon. Cells are first harvested from a patient. To reduce the occurrence of immunological reaction, cells are preferably obtained from the patient who is to receive the artificial tendon. For example, fragments of a tendon disrupted by injury can be surgically removed. After any necessary cell sorting, the tendon or fibroblast cells are placed, together with the matrix, in an appropriate cell culture solution or device. Mechanical action may be employed to ensure the correct orientation of the cells onto the matrix.

The cells are cultured either in a monolayer culture then transferred to a collagen-imbedded culture, or in a matrix material culture using, for instance, RPMI 1640 medium. Serum albumin, or bovine, or human serum may be added. The culture material may also contain any of a variety of additional ingredients, including transferrin, insulin, hydrocortisone, retinoic acid, epidermal growth factor, vascular endothelial growth factors (VEGF-A, VEGF-C, EG-VEGF), insulin growth factor (IGF), keratinocyte growth factor, basic fibrinogen growth factor (b-FGF), acidic growth factor (A-FGF), transforming growth factor alpha (TGF-α), hepatocyte growth factor (HGF), interleukin 8 (IL-8), pleiotropin, ENA-78, Gro-α, sonic hedgehog (Shh), platelet-derived growth factor B (PDGF-B), thrombin (II-a), sphingosine 1-phosphate (SIP), angiopoietin 1 (ANG-1), angiopoietin 2 (ANG-2), ephrins, placental growth factor (PIGF), TGF-β, endothelial inegrins, cadherin, PECAM, angiogenincyclin E, E-cadherin, PD-ECGF, TGF-B 1, HSP-27, glutamide, and nanoscale surface roughness.

Depending on the type and amount of cells required, the culture conditions and reagents employed, the cells may optimally be harvested after a period of 24 hours to 14 days. The fibers should be prepared or chosen so that the cells naturally adhere to the fibers. Preferably, they include fiber surface roughness to stimulate binding. Further, an adhesive stimulant may be used. Adhesive stimulating proteins can be employed, such as FPG, or pronectin, fibronectin, EGF, cadherin, or catectin. Such proteins can be used during the fiber assembly, or as a bath for the structure prior to cellular coating. The cells will orient in response to factors such as media flow direction, electrical current, and gravitational pull. The orientation of the cells is relevant, and the use of specific media for directions, such as unidirectional vs. multidirectional, electrical currents, such as low voltage or longitudinal, and gravitational, such as circumferential, direct, or magnetic, can be used to achieve the desired cellular orientation.

A cellular adherent step can take place in the same media as the cell culture; alternatively, the media may be varied to accelerate adhesion. Cellular adhesion to the fibers generally takes from 24 hours to 14 days. Cellular adhesion can be assessed with a microscope, alternatively, to determine the extent of cellular adhesion, cells remaining in the media may be counted or predicted.

After the desired degree of cell growth, the coated artificial tendon may be implanted into a patient. Physical therapy regimens may be used to ensure the artificial tendon is united with the natural body components and to permit further growth of the cellular coating as necessary. Physical therapy may also be preferred when the artificial tendon is implanted prior to coating with cellular material.

### EXAMPLE III

Fabrication of an Artificial Bone

Due to a variety of internal and external factors, bones may require replacement or augmentation. The present invention provides a fibrous artificial bone matrix that may be invaded by cellular material to recreate or reconstruct natural bone. The addition of cellular material is further described below in Example IV.

Depending on the intended use of the artificial bone, a variety of materials and shapes are provided. For replacement of long, weight-bearing bones such as the femur, a structure made of a combination of fiber and metal is utilized. As seen in FIG. 2, a structural support 20 is comprised of a set of parallel columns 21. Columns 21 are comprised of strong metal or composite that can support the necessary amount of weight, depending on the patient and ultimate use. A biocompatible metal is preferred, alternatively, a non-compatible material may be coated or treated to prevent adverse reaction once implanted into a patient. It may be possible to provide for selected break points in columns 21, to accommodate expansion of structural support 20 and a corresponding lengthening of the artificial bone.

Appropriate metals can include, but are not limited to, aluminum, copper, stainless steel, titanium, platinum, silver, and heavy metals (Cr, Fe, Ni, and Cd). The metals and fibers may be joined through a thermal bond, chemical adhesives such as fibers, plastic, silastic, or chemical adhesive glues, weaving the fibers used alone or in conjunction with thermal or adhesive bonds. In selecting the materials and joining techniques, properties such as strength, density, flexibility, ability to attract cells, ability to support full osteo cell growth, and ability to permit vascularization of the structure are desired. It is preferred that the material selected exhibit the desirable properties under all physiologic stress conditions.

As shown in FIG. 2, columns 21 are configured in a roughly cylindrical shape, and held in place and further supported by circumferential rings 22. Rings 22 may be comprised of metal or fiber, strong yet flexible fiber compositions are preferred. Rings 22 are attached to columns 21 along the length of structural support 20 through means suitable based on the materials employed, for example, soldering, using a chemical adhesive, or tying. The size of the columns and the rings depend on the final use of the artificial bone, for example, a length of from about 0.5 cm to 30 cm and width of from about 0.5 cm to 3.0 cm.

While a generally straight, cylindrical shape is shown, alternate shapes may be required for different applications. While an artificial femur or portion thereof would have a generally straight, cylindrical shape, an artificial rib would have a curved, generally cylindrical shape. An artificial radius or portion thereof would have a somewhat cylindrical shape, tending toward an oval shape and semi-prismatic to correlate to the natural flattened shape with a widening at the distal end. Other embodiments of the artificial bone of the present invention would not be cylindrical in form, rather, they would have an irregular configuration to mimic that of a naturally occurring bone or portion thereof, such as a vertebra or lower jaw.

Prior to implantation, structural support 20 may be treated with a cell growth promoter, it may be lined and/or coated with a cell growth matrix, it may be coated with a cell population, or any combination of these treatments. Examples of an appropriate cell growth promoters include, but are not limited to, fibronectin, transferrin, insulin, hydrocortisone, retinoic acid, epidermal growth factor, vascular endothelial growth factor, insulin growth factor, keratinocyte growth factor, basic fibrinogen growth factor, angiogenincyclin E, E-cadherin, PD-ECGF, TGF-B 1, HSP-27, and glutamine. Physical manipulation such as surface roughness, preferably on the nanoscale level, may also be employed.

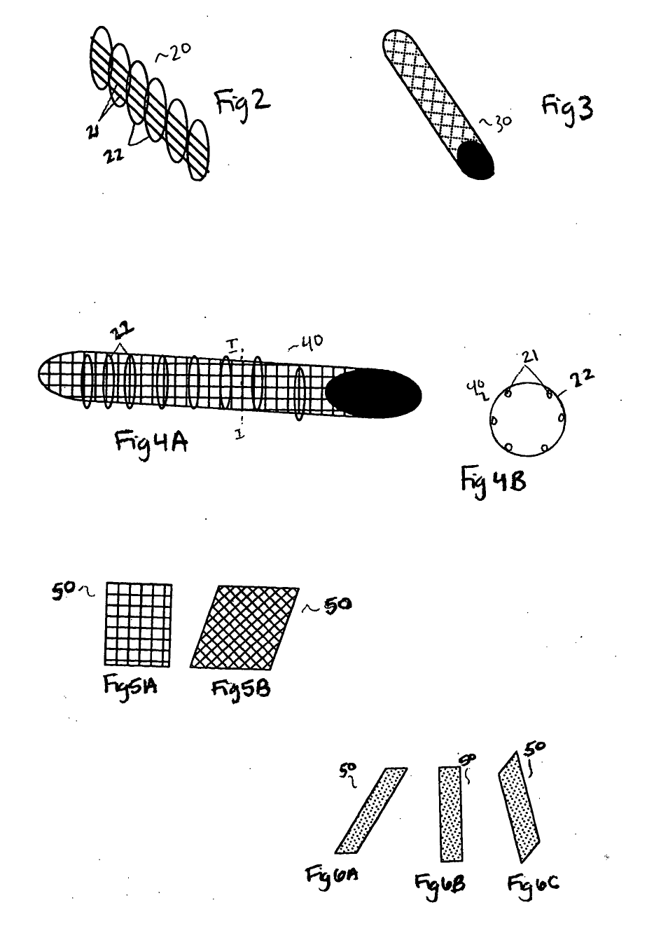

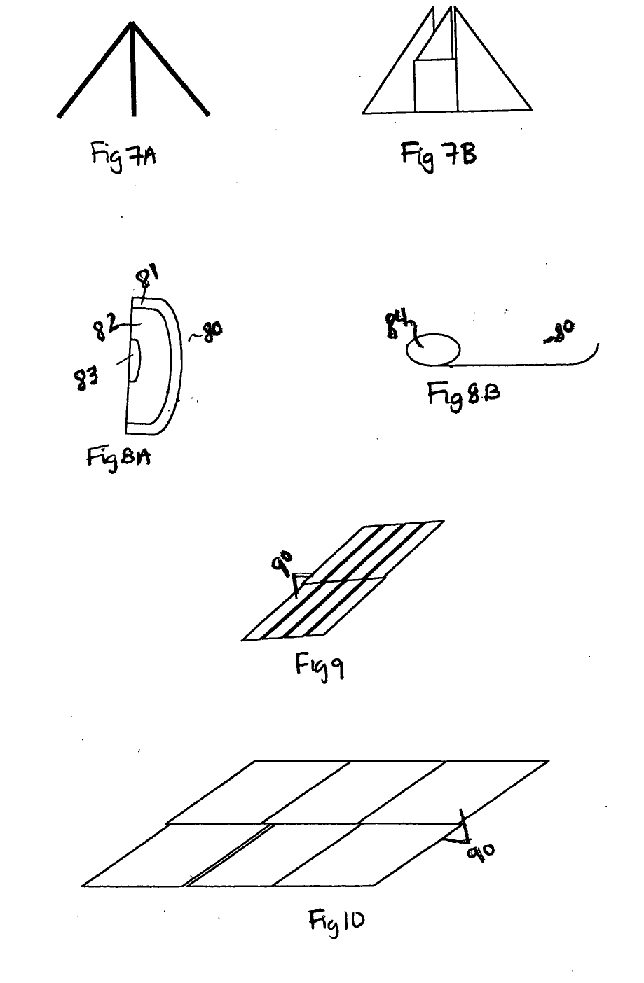

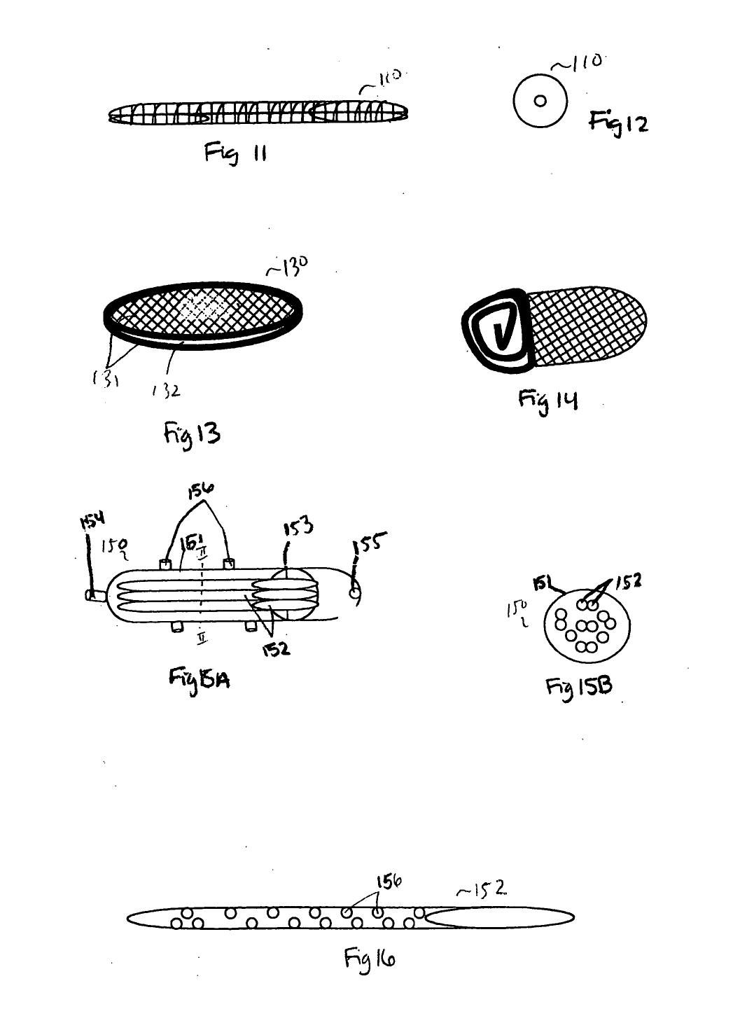

A cell growth matrix 30 is depicted in FIG. 3. Cell growth matrix 30 is configured to correspond to the interior or exterior measurements of a structural support. The material employed may be any biocompatible material in a mesh or mesh-like state, preferably having a pore size of between about 0.1 μm and 1,000 μm to facilitate cell growth. Optionally, cell growth matrix 30 may be made from a digestable or reabsorbable material. Appropriate digestible or absorbable materials include silastic, proline methacrylate, nylon, dacron, polydimethylsiloxane, polyurethane, polyethylene, vicryl, and goretex. In certain embodiments, either or both of the structural support 20 and the cell growth matrix 30 may be configured from silastic, praline, methacrylate, nylon, Dacron, polydimethylsiloxane, polyurethane, polyethylene, vicryl, goretex, polypropylene, octyl-2-cyanoacrylate, polymethylacrylate, polylactide, aramid, polystyrene, poly L-lysine, terephthalate (PET) POLYMER (NEUTRAL), POLYMER (SILICA), polygalctide, hydroxylapatite, latex, rubber, elastic, glass, ceramic, plastic, polyester, aluminum, copper, stainless steel, titanium, platinum/silver, or heavy metals (Cr, Fe, Ni and Cd).

Cell growth matrix 30 may be sized and shaped to fit in or over a structural support without requiring attachment, alternatively, cell growth matrix 30 may be attached to a structural support through, for example, adhesive or tying.

An artificial bone 40 comprised of a matrix-lined structural support is shown in FIG. 4A. Artificial bone 40 has an open, cylindrical shape as shown in the cross section depicted in FIG. 4B. The relative size of columns 21 and rings 22 shown is by way of example, proportions and configurations would necessarily vary depending on the materials selected and the intended use. A hollow core is provided, working in conjunction with openings or pores tangentially located throughout the length to allow vascular adjacent tissue ingrowth. Columns may be placed through the middle and orientated toward the periphery and central region.

In addition to artificial bones for weight-bearing uses, damage or defects in non weight-bearing bones also occur. The present invention provides for replacement bones for these purposes as well. Because the mechanical demands on a non weight-bearing bone are relatively low, the primary structure of this embodiment of an artificial bone is comprised of non-metal fibers. As shown in FIGS. 5A and 5B, an artificial bone mesh 50 may be provided. Rectangular and parallelogram shapes are depicted in FIGS. 5A and 5B, respectively, however, any necessary shape or size may be created. The cross linking of the fibers that comprise artificial bone mesh 50 is provided with a spacing that will achieve the desired strength, flexibility, and shape retention based on particular use and materials employed. For example, a stiff fiber with relatively large diameter could be woven into a mesh with relatively large pore size and relatively high strength, but with low flexibility.

Other appropriate materials for the artificial bones may involve those described above with reference to Example I.

FIGS. 6A, 6B and 6C show alternate configurations of artificial bone mesh 50. The material employed for the embodiments shown is a solid material with well distributed pores. Such a material can be provided with varying thickness to mimic varying contours and varying degrees of flexibility provided by natural bones. FIG. 7A schematically represents a configuration of artificial bone mesh to create a replacement nose. Alternatively, a variety of panels of artificial bone mesh may be joined together, as shown in FIG. 7B, to create a replacement nose. The embodiment depicted in FIG. 7B may be preferred for some applications, because the various components may be arranged to have a similar final appearance as a patient's natural nose. Also, the various artificial bone mesh components can vary in flexibility and strength to better mimic a natural nose and the decrease in strength and increase in flexibility seen between the bridge and tip.

Another application of the non weight-bearing artificial bone of the present invention is the formation of an artificial ear. One example is shown in FIG. 8A, with a view from the side shown in FIG. 8B. Proper selection of artificial bone mesh allows the creation of an artificial ear 80, complete with helix 81, anti-helix 82, and concha 83. As seen in the side view of FIG. 8B, artificial ear 80 is provided with an enlarged bottom 84, similar to the naturally occurring thickening at the lobule. The cartilagenous nature of noses and ears can be replicated by artificial bone meshes such as those depicted in FIGS. 5A-6C. After preparing the artificial bone, a suitable synthetic or natural coating can be added, such as a patch of artificial skin such as described below in Example IV.

### EXAMPLE IV

Deposit of Cellular Coating on an Artificial Bone

Prior to implantation, cells may be grown upon and within an artificial bone such as that described in Example III. In the event that the artificial bone will be used for repair after an injury, bone cells can be harvested from bone fragments collected during surgical debridement. Alternatively, or for other applications, bone cells may be harvested from the illiac crest, sternum, or rib bones. It may be preferable to culture the harvested cells in solution to a particular concentration prior to intermingling the cells and the artificial bone.

The cells and artificial bone are placed in an appropriate container with the necessary reagents and the cells are allowed to coat the surfaces of the artificial bone. Once the desired level of cell growth is reached, the cell-coated artificial bone is implanted in a patient.

Materials and techniques regarding cell culture and cell adhesion are described above with reference to Example II. Conditions for cell culturing on the artificial bone are usually at standard culture temperatures, e.g., 32° C. to 38° C. There can either be no motion provided, or a mild centrifugal or rocking motion. Even when coated with cells, there will preferably be a hollow core to the bone, and openings or pores located tangentially throughout the length which allow for vascular adjacent tissue ingrowth.

For uses such as replacement noses and ears, cartilage cells may be preferred for growth on the selected matrix. In any embodiment, a mixture of bone and cartilage cells may be utilized to impart the necessary structural integrity and strength while permitting the desired level of flexibility.

Because noses and ears are exposed externally, an artificial nose or ear according to the present invention may preferably be coated with skin and/or cutaneous material prior to placement on a patient. This procedure is described in Examples V and VI, below.

### EXAMPLE V

Fabrication of Artificial Skin and Cutaneous Material

Unfortunately, considerable numbers of patients suffer damage to or destruction of skin and cutaneous regions. These sort of injuries or defects can be particularly distressing because, in addition to serious problems with motility and infection, they often directly affect outward appearance. Artificial skin and cutaneous material may be prepared in a variety of ways, depending on the intended use.

A temporary artificial skin may be desired for a smaller injury or where access to underlying structure may be required in the future. A matrix of degradable or reabsorbable fibers can be woven or prepared for implantation and optional pre-implantation coating. A permanent, flexible artificial skin may be preferred that can be used on, for example, hands. This matrix is prepared from biocompatible, non-immunologic material that will not cause scarring or recruit scar tissue.

The artificial skin and cutaneous material may be formed from materials such as silastic, proline, methacrylate, nylon, dacron, polydimethylsiloxane, polyurethane, polyethylene, vicryl, and goretex. Further materials include polypropylene, octyl-2-cyanoacrylate, polymethylacrylate, polylactide, aramid, polystyrene, poly-L-lysine, terephthalate (PET), neutral polymer, silica polymer, polygalctide, and hydroxylapatite. In the formation of artificial skin, it may be preferred to used thermal bonding in the fibers; however, fibrous, plastic, silastic or chemical adhesives or a weaving technique for instance with a knot-lock or knitting fastening style may also be employed.

A further type of artificial skin is comprised of a matrix prepared from biocompatible, stable materials. The fiber comprising the matrix is stronger than that used for other artificial skin purposes, and is woven more densely, yet remains relatively flexible. The matrix forms a constructive base for skin growth and is able to recruit some scar tissue from the patient to form a more solid base. Uses for this matrix of artificial skin include covering for noses or ears, including the artificial nose or ear described in Example III, as well as abdominal uses and any application where a full thickness of skin must be replaced.

The overall shape, thickness, and dimensions of the artificial skin will vary depending on use. A parallelogram shaped patch is preferred for ease of construction and use. A series of patches may be employed depending on the size of surface area to be covered. FIG. 9 shows a series of long, narrow artificial skin patches 90, whereas FIG. 10 shows a series of relatively short, fairly wide artificial skin patches 90. Consideration is given to degree of desired flexibility and scar prevention when assembling the appropriate sizes and shapes of artificial skin patches.

For certain uses, the artificial skin may be fabricated and directly implanted into a patient. This provides for a supportive structure to which native cells may migrate and grow. Some or all of the fibers may be digestible. This form of artificial skin may be coated with subcutaneous cell growth factors and anti-inflammatory agents prior to implantation. For alternative uses, the artificial skin matrix may be coated with cells prior to implantation, as described in Example VI.

Growth factors and other agents may be used to improve the artificial skin and cutaneous material. Such materials include, but are not limited to, fibronectin, transferrin, insulin, hydrocortisone , retinoic acid, epidermal growth factor, vascular endothelial growth factor, insulin growth factor, keratinocyte growth factor, basic fibrinogen growth factor, angiogenincyclinE, E-cadherin, PD-ECGF, TGF-B 1, HSP-27, and glutamine. Surface roughness on the fibers at the nanoscale level may also be used.

### EXAMPLE VI

Deposit of Cellular Coating on Artificial Skin and Cutaneous Material

In order to have cells for coating, cells are harvested from a donor, preferably from the patient who will receive the artificial skin. Alternatively, cells may be obtained from a commercial supplier where available. One method of harvesting cells is a punch biopsy. The material thus obtained can be sorted into superficial cutaneous cells and deep cutaneous cells. These cells can be seeded on an artificial skin matrix such as that described in Example V. Depending on the intended final use, one or both types of cells are seeded and permitted to grow to a desired level of coating. Thereafter, the coated artificial skin is transferred to a patient or used to coat an artificial bone such as an artificial nose, which is later transferred to a patient.

The cellular coating can be deposited through the use of gravity. Cells are placed into a chamber which contains the matrix, and the cells float down onto the matrix through gravitational pull. The cells then adhere to the matrix. The cells used therein can be cultured in a monolayer culture and transferred into a collagen-imbedded culture, or the cells can be cultured in matrix materials using, for instance, RPMI 1640 medium with serum albumin, or with bovine or human serum. The materials may contain additional factors to encourage growth and adhesion. Such additional factors may include transferrin, insulin, hydrocortisone, retinoic acid, epidermal growth factor, vascular endothelial growth factors (VEGF-A, VEGF-C, EG-VEGF), insulin growth factor (IGF), keratinocyte growth factor, basic fibrinogen growth factor (b-FGF), acidic growth factor (A-FGF), transforming growth factor alpha (TGF-α), hepatocyte growth factor (HGF), interleukin 8 (IL-8), pleiotropin, ENA-78, Gro-α, sonic hedgehog (Shh), platelet-derived growth factor B (PDGF-B), thrombin (II-a), sphingosine 1-phosphate (SIP), angiopoietin 1 (ANG-1), angiopoietin 2 (ANG-2), ephrins, placental growth factor (PIGF), TGF-β, endothelial inegrins, cadherins, PECAM, angiogenincyclin E,E-cadherin, PD-ECGF, TGF-B 1, HSP-27, glutamide, and nanoscale surface roughness.

The cell culture duration may be from approximately 24-48 hours to many weeks, such as approximately 23 weeks. Preferred patch sizes are from approximately 1-10 cm, for example, approximately 5 cm. Multiple patches may be employed to provide sufficient surface area for desired skin area coverage. In the case of an artificial nose or ear, adequate skin coverage may include the need for a healthy adjacent or flat area which includes a vascular intact pedical. As with other artificial materials, sterility must be maintained.

### EXAMPLE VII

Implantable Cell Growth Cage

Some medical treatments are accomplished by administering substitutes for naturally produced materials where a patient is unable to produce sufficient amounts of the material. One example is injectable insulin for certain diabetic patients, another example is the oral or subcutaneous administration of hormones to prevent pregnancy. An alternative to ongoing administration treatments is to transplant cells capable of production of the desired material into the patent. These cells, properly transplanted, may be able to provide sufficient materials so that additional supplementation and the associated expense and burden are not required.

An implantable cell growth cage 110 is shown in FIG. 11. The embodiment of FIG. 11 is tube shaped and may be implanted in, for example a vein or artery. Cell growth cage 110 is comprised of porous, biocompatible materials such as woven nylon fiber, polyurethane, polyethylene or plastic. Standard commercially available plastic may be employed. Such a plastic may be lined with a non-reactive material, such as the materials provided in Example I for use with fibers. Prior to implantation, the cell growth cage is coated throughout with cells capable of producing the desired materials.

One way to provide such cells is to harvest them from a patient, for example, using a large bore subcutaneous needle, adipose or fibroblast cells. To assure biocompatibility, use of cells donated from the ultimate recipient of the cell growth cage is preferred. These cells can be genetically transformed to produce one or more or different materials, such as myostatin, follistatin, insulin, parathyroid hormone, pituitary hormone, testosterone, estrogen, progesterone, and thyroid hormone, among others. Numerous references are available describing methods and materials for genetic transformation. Examples of such references are included in the Reference section of the present application. The typical vector used is adeno, or plasmid vectors which do not have a re-infective capability. Once transformed, the cells are allowed to grow in culture. Eventually they are used to form a cellular coating on a cell growth cage. When coated with cells, the outer diameter of a tube shaped cell growth cage such as that in FIG. 11 is between 10 and 16 gauge, with an inner diameter of 0.5-0.05 mm. The nature of the growth cage materials and design provide that the cells generally will not migrate or move.

Culture conditions and materials have been described with reference to previous examples and would be applicable herewith. Cell migration or movement is prevented through the use of selective pore sizes.

A tube shaped cell growth cage can be inserted in to a native vein or artery. As seen in FIG. 12, the cross section of tube shaped cell growth cage 110 permits blood flow through the central portion, the blood flowing through this portion will nourish the cells in cell growth cage 110 and collect materials produced therefrom. This vascular conduit-type tube is located similarly to commercial vascular vessel grafts, by operative suture into position.

An alternative to arterial or venous implantation could be made with a disk shaped cell growth cage 130, as shown in FIG. 13. Disk shaped cell growth cage 130 is comprised of a fibrous matrix, for example a nylon mesh. Two similarly sized disks 131 are joined along a portion of their borders. Thus, an opening 132 is provided along a portion of disk shaped cell growth cage 130. Optionally, disk shaped cell growth cage 130 may be woven or molded directly as a one-piece unit. This configuration provides surface area for the growth of desired cells. Transfected cells are cultured around the matrix structure of disk shaped cell growth cage 130 prior to transplant. Once suitable cell growth is accomplished, the disk shaped cell growth cage 130 has a width of 25-100 mm, a diameter of 1-3 cm and a length of 3-6 cm.

One way implantation of a seeded disk shaped cell growth cage 130 can be accomplished is by tightly rolling the disk and inserting it into a carbohydrate or other digestable capsule, such as those produced by Capsugel, Greenwood, S.C. The capsule is then loaded in to a 10 or 16 gauge needle and injected into the desired region of a patient. As the capsule dissolves, the disk unrolls into its original shape. For this method of insertion, a disk comprised of fiber that is flexible and has a ‘memory’ is preferred. Once established inside a patient, blood may flow in and around the disk, providing nutrients to the seeded cells and removing the manufactured materials to the rest of the body.

Fibers disclosed previously can be employed to produce the disk-shaped cell growth cage, with preference given to highly flexible fibers. Blood flow does not perfuse the disk; however, fluid from extra-cellular, extra-vascular compartments reaches the disk by osmosis.

As noted above, pore size is relevant in order to prevent cell migration or movement. Examples or appropriate pore sizes include, for the implantable disk type, such as flat, circular, or tubular disk, a pore size of 0.01 μm to 0.2 μm can restrict large protein such as albumin and all cells. For an implantable vascular conduit, a pore size of 0.01 μm to 0.2 μm is also employed. In the flexible worm-like envelope, a pore size of 0.01 μm is preferred.

### EXAMPLE VIII

Cell Growth Chamber

The present invention relies in part upon cultured cells and/or cultured transfected cells. A chamber that facilitates cell growth is also provided as shown in FIG. 15A. The cell growth chamber 150 comprises a central portion 151 containing a plurality of inner tubes 152. A diameter may range from about 1 to 5 cm, with a length of about 5 cm to about 10 cm. Inner tubes 152 are comprised of a material that is inert with respect to the cells cultured. Approximately 12-20 inner tubes 152 are typically provided within one cell growth chamber 150.

Cells for culture are placed within inner tubes 152 and loaded into the body 151 of a cell growth chamber 150. The embodiment depicted in FIG. 15A has an opening 153 that permits a user to pull cell growth chamber 150 apart into two parts. Other options, including smaller openings, are within the spirit of the present invention. The opening should be capable of forming a seal when closed to avoid loss of liquid from the chamber or entry of atmospheric gases into the container. Once inner tubes 152 containing cells to be cultured are loaded within cell growth chamber 150, the appropriate cell culture solution is added to cell growth chamber through entry flow connector 154. Entry flow connector 154, as well as other connectors, are ports that provide access to the chamber but are capable of being sealed or closed when not in use. After passing through cell growth chamber 150, culture solution exits through an exit flow connector 155. Side flow entry and exit tubes 156 are provided on body 151 of cell growth chamber 150 for example, for administration of supplemental nutrients or to test for cell growth parameters.

FIG. 15B shows a cross-sectional view of cell growth chamber 150. A more detailed view of an inner tube 152 is shown in FIG. 16. Pores 156 are provided along the length of inner tube 152 to provide additional circulation of nutrients and to facilitate waste or cell product removal from the cultured cells. The pores permit significant circulation by fluid osmotic forces, but restrict cell and macro-molecular sized proteins/carbohydrates/lipids from passing through. Examples of restricted materials include albumin, immunoglobulins, and antibodies. Pore sizes may range from 0.01 μm to 0.2 μm.

One preferred use for the cell growth chamber is to culture transfected animal cells. Cells such as fibroblasts, vascuar smooth muscle or other muscle cells, adipose cells or chinese hamster ovary cells are transfected with a desired gene such as one encoding myostatin, including T myostatin-1, T myostatin-2, bovine myostatin, mouse myostatin, and human myostatin, or follistatin genes. The transfected cells are placed in the cell growth chamber and the desired products are isolated from the materials removed through the exit flow tubes. Proteins thus produced may be encased in a digestible capsule and administered orally. The protein may be bound to a polysaccharide to allow easy absorption.

A harvest needle that may be employed with the present invention includes any standard size needle, ranging from 10 to 14 gauge needles. A harvest needle can be manufactured of stainless steel, or other standard material employed in the manufacture of needles. Preferred lengths range from 3 cm to 10 cm. In order to perform the harvesting function, the needle can be provided with a cutting bevel edge, which is blocked with a solid insert guide, the guide being removable upon entry into the tissue of interest. Aspiration is applied until the desired depth is reached, the needle with the inner harvested material is then removed, and the tissue is flushed from the harvest needle with a harvest solution such as saline. Additional tissue harvesting can be performed immediately, possibly with the same needle.

The foregoing description and examples have been set forth merely to illustrate the invention and are not intended to be limiting. Since modifications of the disclosed embodiments incorporating the spirit and substance of the invention may occur to persons skilled in the art, the invention should be construed broadly to include all variations falling within the scope of the appended claims and equivalents thereof.

### REFERENCES

1: Reisdorff J, En-Nia A, Stefanidis I, Floege J, Lovett D H, Mertens P R.
   Transcription factor ets-1 regulates gelatinase a gene expression in mesangial cells.
   J Am Soc Nephrol. 2002 June; 13(6):1568-78.
2: Tsutsumi S, Tomisato W, Hoshino T, Tsuchiya T, Mizushima T.
   Transforming Growth Factor-betal Is Responsible for Maturation-Dependent Spontaneous Apoptosis of Cultured Gastric Pit Cells.
   Exp Biol Med (Maywood). 2002. June; 227(6):402-11.
3: Van Der Meijden C M, Lapointe D S, Luong M X, Peric-Hupkes D, Cho B, Stein J L, Van Wiinen A L, Stein G S.
   Gene Profiling of Cell Cycle Progression through S-Phase Reveals Sequential Expression of Genes Required for DNA Replication and Nucleosome Assembly.
   Cancer Res. 2002 Jun. 1; 62(11):3233-43.
4: Biodegradable, Elastic Shaped-memory polymers for potential Biomedical Applications. Lendlein, Andrea, Langer, Robert Science 296:2002. pg. 1673-1676
6: Bai X C, Luo S O, Bai J, Deng F Zheng WS, Ji OS.
   Up-regulation of Phosphatidylinositol-3 Kinase Signaling in plcgl Gene Null Fibroblasts.
   Sheng Wu Hua Xue Yu Sheng Wu Wu Li Xue Bao (Shanghai). 2001; 33(6):677-681.
   PMID: 12035061 [PubMed—as supplied by publisher]
7: Kim D S, Kim S Y, Chung J H. Kim K H. Eun H C, Park K C.
   Delayed ERK activation by ceramide reduces melanin synthesis in human melanocytes.
   Cell Signal. 2002 September; 14(9):779-785.
8: Prietzsch H, Brock J, Kleine H D, Liebe S, Jaster R.
   Interferon-alpha inhibits cell cycle progression by Ba/F3 cells through the antagonisation of interleukin-3 effects on key regulators of G(1)/S transition.
   Cell Signal. 2002 September; 14(9):751-9.
10: Gupta S, Afag F, Mukhtar H.
   Involvement of nuclear factor-kappa B, Bax and Bcl-2 in induction of cell cycle arrest and apoptosis by apigenin in human prostate carcinoma cells.
   Oncogene. 2002 May 23; 21(23):3727-38.
11: Gupta S, Sen S.
   Myotrophin-kappaB DNA interaction in the initiation process of cardiac hypertrophy.
   Biochim Biophys Acta. 2002 May 8; 1589(3):247-60.
12: Wang H S, Wasa M, Okada A.
   Amino acid transport in a human neuroblastoma cell line is regulated by the type I insulin-like growth factor receptor.
   Life Sci. 2002 May 31; 71(2):127-137.
13: Paski S C, Xu Z.
   Labile intracellular zinc is associated with 3T3 cell growth.
   J Nutr Biochem. 2001 November; 12(11):655-661.
14: Spinella F, Rosano L, Di Castro V, Natali P G, Ba nag to A.
   Endothelin-1 induces vascular endothelial growth factor by increasing hypoxia-inducible factor-1 alpha in ovarian carcinoma cells.
   J Biol Chem. 2002 May 22 [epub ahead of print]
18: Uchimiva H, Furukawa T, Okamoto M, Nakajima Y, Matsushita S, Ikeda R, Gotanda T, Haraguchi M, Sumizawa T, Ono M, Kuwano M, Kanzaki T, Akiyama S.
   Suppression of thymidine phosphorylase-mediated angiogenesis and tumor growth by 2-deoxy-L-ribose. Cancer Res. 2002 May 15; 62(10):2834-9.
19: Parekh T V, Gama P, Wen X, Demopoulos R, Munger J S, Carcangiu M L, Reiss M, Gold L I.
   Transforming growth factor beta signaling is disabled early in human endometrial carcinogenesis concomitant with loss of growth inhibition.
   Cancer Res. 2002 May 15; 62(10):2778-90.
1: Lui V W, Grandis J R.
   EGFR-mediated cell cycle regulation.
   Anticancer Res. 2002 January-Febuary; 22(1A):1-11.
23: Hopfner M, Berger A, Folsch U R, Loser C.
   Effects of Insulin-Like Growth Factor I on Growth and Polyamine Metabolism in Various
   Organs in Rats. Digestion. 2002; 65(2):103-111.
24: Schiemann W P, Blobe G C, Kalume D E, Pandey A, Lodish H F.
   Context-specific effects of fibulin-5 (DANCE/EVEC) on cell proliferation, motility, and invasion: Fibulin5 is induced by TGF-beta and affects protein kinase cascades.
   J Biol Chem. 2002 May 20 [epub ahead of print]
28: Ling M T, Wang X, Tsao S W, Wong Y C.
   Down-regulation of Id-1 expression is associated with TGFbetal-induced growth arrest in prostate epithelial cells.
   Biochim Biophys Acta. 2002 Apr. 15; 1570(3):145-52.
29: Tokunaga Y, Hosogi H, Hoppou T, Nakagami M, Tokuka A, Ohsumi K.
   Prognostic value of thymidine phosphorylase/platelet-derived endothelial cell growth factor in advanced colorectal cancer after surgery: evaluation with a new monoclonal antibody.
   Surgery. 2002 May; 131(5):541-7.
33: Ellis P E, Wong Te Fong L F, Rolfe K J, Crow J C, Reid W M, Davidson T, MacLean A B, Perret CW.
   The role of vascular endothelial growth factor-A (VEGF-A) and platelet-derived endothelial cell growth factor/thymidine phosphorylase (PD-ECGF/TP) in Paget's disease of the vulva and breast.
   Anticancer Res. 2002 March-April; 22(2A):857-61.
37: London S J, Yuan J M, Travlos G S, Gao Y T, Wilson R E, Ross R K, Yu M C.
   Insulin-Like Growth Factor I, IGF-Binding Protein 3, and Lung Cancer Risk in a Prospective Study of Men in China.
   J Natl Cancer Inst. 2002 May 15; 94(10):749-54.
38: Diep O N, El Mabrouk M, Cohn J S, Endemann D, Amiri F, Virdis A, Neves M F, Schiffrin E L.
   Structure, endothelial finction, cell growth, and inflammation in blood vessels of angiotensin II-infused rats: role of peroxisome proliferator-activated receptor-gamma.
   Circulation. 2002 May 14; 105(19):2296-302.
40: Oiang Y W, Kopantzev E, Rudikoff S.
   Insulinlike growth factor-I signaling in multiple myeloma: downstream elements, finctional correlates, and pathway cross-talk.
   Blood. 2002 Jun 1; 99(11):4138-46.
41: Armstrong R J, Hurelbrink C B, Tyers P, Ratcliffe E L, Richards A. Dunnett S B, Rosser A E, Barker RA.
   The potential for circuit reconstruction by expanded neural precursor cells explored through porcine xenografts in a rat model of Parkinson's disease.
   Exp Neurol. 2002 May; 175(1):98-111.
42: Moustakas A, Pardali K, Gaal A, Heldin C H.
   Mechanisms of TGF-beta signaling in regulation of cell growth and differentiation.
   Immunol Lett. 2002 Jun. 3; 82(1-2):85-91.
44: Xie Y, Sproule T, Li Y, Powell H, Lannutti J J, Kniss D A.
   Nanoscale modifications of PET polymer surfaces via oxygen-plasma discharge yield minimal changes in attachment and growth of mammalian epithelial and mesenchymal cells in vitro.
   J Biomed Mater Res. 2002 August; 61(2):234-45.
47: Nagai S. Washivama K, Kurimoto M, Takaku A, Endo S, Kumanishi T.
   Aberrant nuclear factor-kappaB activity and its participation in the growth of human malignant astrocytoma.
   J Neurosurg. 2002 May; 96(5):909-17.
48: Kaji T, Fujiwara Y, Inomata Y, Hamada C, Yamamoto C, Shimada S, Lee J B, Hayashi T.
   Repair of wounded monolayers of cultured bovine aortic endothelial cells is inhibited by calcium spirulan, a novel sulfated polysaccharide isolated from Spirulina platensis.
   Life Sci. 2002 Mar. 8; 70(16):1841-8:
49: Voelkel N F, Cool C, Taraceviene-Stewart L, Geraci M W, Yeager M, Bull T, Kasper M, Tuder R M.
   Janus face of vascular endothelial growth factor: the obligatory survival factor for lung vascular endothelium controls precapillary artery remodeling in severe pulmonary hypertension.
   Crit Care Med. 2002 May; 30(5 Suppl):S251-6. Review.
52: Lawler J.
   Thrombospondin-1 as an endogenous inhibitor of angiogenesis and tumor growth.
   J Cell Mol Med. 2002 January-March; 6(1):1-12.
53: Kimura T, Majima Y, Guo Y, Yoshida T.
   The effect of growth factors on the proliferation and differentiation of human nasal gland cells. Arch Otolaryngol Head Neck Surg. 2002 May; 128(5):578-82.
55: Pavlin D, Gluhak-Heinrich J.
   Effect of mechanical loading on periodontal cells.
   Crit Rev Oral Biol Med. 2001; 12(5):414-24. Review.
56: Orpana A, Salven P.
   Angiogenic and lymphangiogenic molecules in hematological malignancies.
   Leuk Lymphoma. 2002 Febuary; 43(2):219-24.
57: Crespo J L, Powers T, Fowler B, Hall M N.
   The TOR-controlled transcription activators GLN3, RTG1, and RTG3 are regulated in response to intracellular levels of glutamine.
   Proc Natl Acad Sci U S A. 2002 May 14; 99(10): 6784-9.
58: Teng J, Wang Z Y, Biorling D E.
   Estrogen-induced proliferation of urothelial cells is modulated by nerve growth factor.
   Am J Physiol Renal Physiol. 2002 June; 282(6): F1075-83.
59: Arch Otolaryngol Head Neck Surg 2002 May; 128(5):578-82
   The effect of growth factors on the proliferation and differentiation of human nasal gland cells. Kimura T, Majima Y, Guo Y, Yoshida T.
   Department of Otorhinolaryngology, Mie University School of Medicine, 2-174 Edobashi, Tsu, Mie 5148507, Japan.
   OBJECTIVE: To elucidate a mechanism of proliferation and differentiation of nasal gland cells, we established a serum-free 3-dimensional culture system for human nasal gland (HNG) cells and examined the effects of epidermal growth factor, keratinocyte growth factor, and retinoic acid on proliferation and differentiation of cultured HNG cells. MATERIALS AND METHODS: Nasal polyps were obtained from patients undergoing endoscopic endonasal sinus surgery. The HNG cells were cultured under a monolayer culture and transferred to a collagen-embedded culture using RPMI 1640 medium containing transferrin, insulin, hydrocortisone, retinoic acid, epidermal growth factor, and keratinocyte growth factor. Cell growth was measured by bromodeoxyuridine incorporation assays. To measure cell differentiation, the percentage of cells containing secretory granules, which were stained with Alcian blue in cytoplasm, was determined. RESULTS: In the serum-free 3-dimensional culture, the HNG cells showed ductal structures containing secretory products in a lumen. The addition of epidermal growth factor promoted the proliferation of HNG cells in its optimal concentrations, and keratinocyte growth factor also enhanced the proliferation of HNG cells. Conversely, the differentiation of HNG cells was not dependent on epidermal growth factor and keratinocyte growth factor. Retinoic acid suppressed the proliferation, but promoted the differentiation of HNG cells. CONCLUSION: Our culture system could be useful for studying the effects of various growth factors and cytokines on HNG proliferation and differentiation to better understand the mechanisms of growth and morphogenesis of nasal glands.
60: J Biomed Mater Res 2002 August; 61(2):234-45
   Nanoscale modifications of PET polymer surfaces via oxygen-plasma discharge yield minimal changes in attachment and growth of mammalian epithelial and mesenchymal cells in vitro. Xie Y, Sproule T, Li Y, Powell H, Lannutti J J, Kniss D A. Department of Obstetrics and Gynecology, Laboratory of Perinatal Research, The Ohio State University, College of Medicine and Public Health, 1654 Upham Drive, Means Hall, Columbus, Ohio 43210. Surface topography is believed to be a factor affecting cellular morphology, proliferation, and differentiation. The effect of surface roughness in the micron to supramicron range has been investigated previously. In the current study, the influence of nanoscale surface roughness was examined in terms of its effects on morphology, cytoskeleton expression, proliferation, differentiation, and apoptosis of three model cell types. Polyethylene terephthalate (PET) disks were etched using oxygen plasma to produce uniform and decidedly nanoscale levels of surface roughness. Three distinct types of cell lines-mouse 3T3-L1 preadipocytes, human JEG-3 choriocarcinoma cells, and human MCF-7 breast adenocarcinoma cells-were cultured on the plasma-treated disks. Untreated PET disks were used as a control. Cytoskeletal proteins (Factin and cytokeratin) exhibited similar patterns of expression. Cell morphology also was similar on both surfaces. Cell growth kinetics for the three types of cells and hormone secretion from the JEG-3 cells were not significantly different from that of the controls (p>0.05). However, the differentiation of preadipocyte 3T3-L1 cells into lipid-laden fat cells was modestly affected by nanoscale surface topography. In addition, 15-deoxy-Delta(12,14)-prostaglandin J(2) (15dPGJ(2))-induced apoptosis of the JEG-3 and MCF-7 cells revealed differences between the two surfaces. Plasma-treated surfaces showed more differentiated and apoptotic cells, respectively, compared to the controls. These results indicate that nanoscale roughness contributes in only moderate ways to cellular adhesion, proliferation, and differentiation in the cell lines tested. Copyright 2002 Wiley Periodicals, Inc. J Biomed Mater Res 61: 234-245,2002
61: Anal Bioanal Chem2002 June; 373(3):190-4
   Estimation of environmental mobility of heavy metals using a sequential leaching of particulate material emitted from an opencast chrome mine complex.
   Poykio R, Peramaki P, Valimaki I, Kuokkanen T.
   Meri-Lappi Institute, Centre for Environmental Technology, University of Oulu, Tietokatu 6, 94600 Kemi, Finland, risto.poykio@kemi.fi
   A four-stage sequential leaching procedure was applied to assess the bioavailability and environmental mobility of heavy metals (Cr, Fe, Cu, Ni and Cd) in total suspended particulate (TSP) material emitted from an opencast chrome mine complex (Kemi, Northern Finland). TSP material was collected on glass fibre filters by a high-volume sampler, and a sequential leaching procedure was used to determine the distribution of heavy metals between the water-soluble fraction (H(2)0), environmentally mobile fraction (CH(3)000NH(4)), the fraction bound to carbonate and oxides (HONH(3)Cl+CH(3)000H), and the fraction bound to silicates and organic matter, that is the environmentally immobile fraction (HNO(3)+HIT +HCI). The sequential leaching procedure was also applied to the certified reference materials VKI (QC Loam Soil A) and PACS-2 (Marine Sediment) to evaluate the accuracy and reproducibility of the leaching procedure. The heavy metals were determined by graphite furnace atomic absorption spectrometry (GFAAS) and flame atomic absorption spectrometry (FAAS). The concentrations of metals in the watersoluble fraction (H(2)0) decreased in the order Fe>Cu>Cr>Ni>Cd, and in the environmentally mobile fraction (CH(3)000NH(4)) in the order Cu>Fe>Ni>Cr>Cd.
62: J Vasc Surg2002 June; 35(6):1260-3
   Comparison of the resistance to infection of rifampin-bonded gelatin-sealed and silver/collagen-coated polyester prostheses.
   Goeau-Brissonniere O A, Fabre D, Leflon-Guibout V, Di Centa I, Nicolas-Chanoine M H, Coggia M. Departments of Vascular Surgery and Microbiology, Ambroise Pare University Hospital and Faculte de Medecine Paris-Ouest, Rene Descartes University. PURPOSE: The purpose of this study was to compare the efficacy of rifampin-bonded gelatin-sealed and silver acetate/collagen-coated knitted polyester prostheses for the prevention of bacteremic graft infection in an animal model. METHODS: Eighteen 6.0-mm polyester grafts (length, 5.0 cm) were implanted in dogs end-to-end into the infrarenal aorta. The dogs were divided into four groups as a function of type of prosthesis implanted. The dogs in groups I (n=3) and II (n=3) received control gelatin-sealed or collagencoated polyester prostheses, respectively. In group III (n=6), the dogs received rifampin-bonded gelatinsealed polyester prostheses. In group IV (n=6), the dogs received silver/collagen-coated polyester prostheses. Two days after implantation, the grafts were challenged with 6×10(9) Staphylococcus aureus intravenously. One week after implantation, the grafts were harvested with sterile technique. Quantitative cultures were obtained from all the harvested grafts. The results were expressed as colony-forming units per cm(2) of graft material. Bacteriologic study was also performed on various tissue samples. The chi(2) test was used to compare the culture proven infection of control and antimicrobial grafts. RESULTS: All the control grafts were infected with S aureus at the time of removal. Five of the six silver/collagen-coated grafts were infected, whereas none of the six rifampin-bonded gelatin-sealed grafts grew S aureus (P<0.01). There was no significant difference in the number of positive culture results of organ samples between the different groups of dogs. CONCLUSION: These results indicate that rifampin-bonded gelatin-sealed polyester grafts are significantly more resistant to bacteremic infection than are silver/collagen-coated polyester grafts in a highly challenging model.
63: Clin Orthop2002 Febuary; (395):11-22
   Bioactive materials in orthopaedic surgery: overview and regulatory considerations. Bauer T W, Smith S T.
   Department of Pathology, The Cleveland Clinic Foundation Cleveland, Ohio 44195, USA. osteoclast@aol.com
   Although bone graft continues to be the standard against which other skeletal substitutes are measured, orthopedic surgeons soon will have various new tools available for skeletal reconstruction. With these tools, the distinctions between inert materials, resorbables, bioactive materials, transplantable tissues, engineered tissues, drugs, and composites become indistinct. Although almost any implanted material evokes some type of host reaction, in the context of reconstructive orthopaedic surgery, bioactive materials can be considered osteogenic, osteoconductive, osteoinductive, or a combination thereof. In the United States, the regulatory control of a new skeletal substitute material is complex, and is based in part on whether the material is considered primarily a biologic, a drug, or a medical device. Different agencies within the Food and Drug Administration have responsibility for regulatory control of different types of products. Although some new materials can be approved by a Premarket Notification (510(K)), others require a Premarket Approval Application. Regulations are being developed that affect the extent of regulatory influence over minimally manipulated tissues for transplantation.
64: J Biomed Mater Res2002 Febuary; 59(2):340-8
   Bioactive sol-gel foams for tissue repair. Sepulveda P, Jones J R, Hench L L.
   Centre for Tissue Engineering and Repair, Department of Materials, Imperial College of Science, Technology and Medicine, Prince Consort Road, London SW7 2BP, United Kingdom. Pilarsi@(net.ipen.br Bioactive glasses are known to have the ability to regenerate bone, but their use has been restricted mainly to powder, granules, or small monoliths. This work reports on the development of sol-gel foams with potential applications as bone graft implants or as templates for the in vitro synthesis of bone tissue for transplantation. These bioactive foams exhibit a hierarchical structure with interconnected macropores (10500 microm) and a mesoporous framework typical of gel-glasses (pores of 2-50 nm). The macroporous matrixes were produced through a novel route that comprises foaming of sol-gel systems. Three glass systems were tested to verify the applicability of this manufacturing route, namely SiO(2), SiO(2)-CaO, and SiO(2)-CaO—P(2)0(5). This new class of material combines large pores to support vascularization and 3-D tissue growth with the ability that bioactive materials have to provide bone-bonding and controlled release of ionic biologic stimuli to promote bone cell proliferation by gene activation. Copyright 2001 John Wiley & Sons, Inc. J Biomed Mater Res 59: 340-348, 2002
65: Adv Dent Res 1999 June; 13:27-33
   The biologic tissue responses to uncoated and coated implanted biomaterials. Steflik D E, Corpe R S, Young T R, Sisk A L, Parr G R.
   Section of Orthopaedic Surgery, Department of Surgery, School of Medicine, Medical College of Georgia, Augusta, Ga. 30912-4030, USA.
   Ultrastructural examination of the morphology and morphometry of the bone supporting uncoated titanium and ceramic implants was assessed in an experimental animal model involving 120 implants placed into the mandibles of 30 adult mongrel dogs. Further, preliminary morphologic and morphometric observations of the bone supporting uncoated and hydroxylapatite-coated endosteal titanium implants was evaluated in a second investigation involving 72 implants placed into the mandibles and maxillae of 6 additional dogs. A densely mineralized collagen fiber matrix was observed directly interfacing with uncoated implants. The only material interposed between the implant and bone matrix was a 20- to 50-mn electron-dense material suggestive of a proteoglycan. Also seen in these same osseointegrated implants were narrow unmineralized zones interposed between the implant and bone matrix. In these zones of remodeling bone, numerous osteoblasts were observed interacting with the collagen fiber matrix. It was shown that a normal homeostasis of anabolic osteoblastic activity and catabolic osteoclastic activity resulted in bone remodeling and the resultant osseointegration of the implants. Hydroxylapatite-coated implants intimately interfaced with healthy bone. The mineralized matrix extended into the microporosity of the HA coating. This matrix contained viable osteocytes.
66: Tissue Eng 2001 February; 7(1):23-33
   Biodegradable polymer scaffolds with well-defined interconnected spherical pore network. Ma P X, Choi J W. Department of Biologic and Material Sciences, Macromolecular Science and Engineering Center, University of Michigan, Ann Arbor, Mich. 48109-1078, USA.mapx@umich.edu Scaffolding plays pivotal role in tissue engineering. In this work, a novel processing technique has been developed to create three-dimensional biodegradable polymer scaffolds with well-controlled interconnected spherical pores. Paraffin spheres were fabricated with a dispersion method, and were bonded together through a heat treatment to form a three-dimensional assembly in a mold. Biodegradable polymers such as PLLA and PLGA were dissolved in a solvent and cast onto the paraffin sphere assembly. After dissolving the paraffin, a porous polymer scaffold was formed. The fabrication parameters were studied in relation to the pore shape, interpore connectivity, pore wall morphology, and mechanical properties of the polymer scaffolds. The compressive modulus of the scaffolds decreased with increasing porosity. Longer heat treatment time of the paraffin spheres resulted in larger openings between the pores of the scaffolds. Foams of smaller pore size (100-200 microm) resulted in significantly lower compressive modulus than that of larger pore sizes (250-350 or 420-500 microm). The PLLA foams had a skeletal structure consisting of small platelets, whereas PLGA foams had homogeneous skeletal structure. The new processing technique can tailor the polymer scaffolds for a variety of potential tissue engineering applications because of the well-controlled architecture, interpore connectivity, and mechanical properties.
67: Ann N Y Acad Sci 2001 November; 944:271-6
   In vitro test of new biomaterials for the development of a bioartificial pancreas. Lembert N, Petersen P, Wesche J, Zschocke P, Enderle A, Doser M, Planck H, Becker H D, Ammon H P.
   Institute of Pharmaceutical Sciences, University of Tubingen, Germany.nicolas.lembert@uni-tuebingen.de The implantation of macroencapsulated islets has the potential to restore endogenous insulin secretion in type 1 diabetics, with no need for lifetime immunosuppression. To match the physiological fluctuations of blood glucose concentrations with appropriate insulin release, the macroencapsulation material must combine immunoprotection with optimal diffusion properties for glucose and insulin. The impact of chemical modifications of polysulphone (PSU) capillary polymers with a cutoff of 50 kD on glucoseinduced insulin secretion of macroencapsulated rat islets was studied in perifusion experiments. The insulin release of free-floating islets showed the typical rapid response to glucose stimulation. Total insulin release (AUC between minute 30 and 120 of perifusion) reached 117+/−22 ng/ml. Blending PSU with polyvinylpyrrolidone or sodium-dodecyl-sulfate was not suitable for islet macroencapsulation, since glucose-induced insulin release was absent or disturbed. Hydroxy-methylation (CH20H) of PSU improved the secretory behavior of macroencapsulated islets depending on the degree of PSU substitution (DS 0.8, AUC 62+/−15 ng/ml; DS 1.8, 111+/−24 ng/ml). In highly substituted PSU-capillaries the kinetics of glucose-induced insulin release was very similar to that observed in free-floating islets. Two consecutive glucose stimulations potentiated insulin release of free-floating islets during the second period of stimulation. Furthermore, freshly isolated macroencapsulated islets responded with more efficient insulin secretion after the initial priming. In conclusion, in vitro membrane screening identified highly substituted hydroxy-methylated PSU as the material of choice for islet encapsulation in a bioartificial pancreas.
68: Science 2001 Nov. 23; 294(5547):1684-8
   Purification of polymeric biomaterials.
   Wandrey C, Vidal D S.
   Department of Chemistry, Swiss Federal Institute of Technology, Lausanne.Christine.Wandrey@epfl.ch Employing a combined filtration and precipitation method, the endotoxin concentration in sodium alginate (SA) and sodium cellulose sulfate (SCS) was reduced to a value of 200 EU/g polymer. This is one tenth of the regulatory threshold calculated, for example, for an appropriate bioartificial pancreas that consists of approximately 420,000 encapsulated islets of Langerhans. The low endotoxin (ET) levels were maintained below this threshold during a six-month storage period. The purification procedure of the polymers did not negatively influence the final microcapsule properties. The mechanical stability of microcapsules from purified material is even slightly higher than that of microcapsules from the original polymers. A second approach to avoid endotoxin release from the device is its direct complexation during the bead or capsule formation process. The durability of endotoxin binding in binary, ternary, and quaternary complexes could be demonstrated for storage in culture medium and saline. Very low total endotoxin release from the complexes was detected after three months in culture medium and five months in saline. This complexation is primarily based on electrostatic interactions with the participating cationic components and provides additional security for the final bioartificial organ or delivery device.
69: Science. 2001 Nov. 23:294(5547):1635-7.
   Self-assembly and mineralization of peptide-amphiphile nanofibers.
   Hartgerink J D, Beniash E, Stupp S I.
   Department of Materials Science and Engineering, Medical School, Northwestern University, 2225 North Campus Drive, Evanston, Ill. 60208, USA.
   We have used the pH-induced self-assembly of a peptide-amphiphile to make a nanostructured fibrous scaffold reminiscent of extracellular matrix. The design of this peptide-amphiphile allows the nanofibers to be reversibly cross-linked to enhance or decrease their structural integrity. After cross-linking, the fibers are able to direct mineralization of hydroxyapatite to form a composite material in which the crystallographic c axes of hydroxyapatite are aligned with the long axes of the fibers. This alignment is the same as that observed between collagen fibrils and hydroxyapatite crystals in bone.
70: J Control Release 2000 Feb. 14; 64(1-3):81-90
   Matrices for tissue engineering-scaffold structure for a bioartificial liver support system.
   Mayer J, Karamuk E, Akaike T, Wintermantel E.
   Chair of Biocompatible Material Science and Engineering, Wagistrasse 23, CH 9852 Schlieren, ETH, Zurich, Switzerland. mayer@biocomp.mat.ethz.ch
   This study proposes a new composite scaffold system. A woven polyethylenterephtalate (PET) fabric was coated on one side with a biodegradable PLGA film, in order to obtain a geometrically polarized scaffold structure for an bioartificial liver support system. The composite structure ensures the stability of the membrane during degradation of the membrane polymer. The mesh size of the composite does not significantly influence the degradation behavior. Hepatocyte culturing studies reveal that the formation of aggregates depends on the mesh size and on the pretreatment: The largest aggregates could be observed after 48 h when PVLA coating, large mesh size and EGF were combined. Thus, the combination of a geometrically structured, partially degradable scaffold with receptor-mediated cell attachment sites offers promising possibilities in liver tissue engineering.
71: Groth T, Seifert B, Malsch G, Albrecht W, Paul D. Kostadinova A, Krasteva N, Altankov G.
   Interaction of human skin fibroblasts with moderate wettable polyacrylonitrile-copolymer membranes. J Biomed Mater Res. 2002 August; 61(2):290-300.
   PMID: 12007210 [PubMed—in process]
72: Oka M.
   Biomechanics and repair of articular cartilage. J Orthop Sci. 2001; 6(5):448-56.
73: Lembert N, Petersen P, Wesche J, Zschocke P, Enderle A, Doser M, Planck H, Becker H D, Ammon H P.
   In vitro test of new biomaterials for the development of a bioartificial pancreas. Ann N Y Acad Sci. 2001 November; 944:271-6.
74: Wandrev C, Vidal D S.
   Purification of polymeric biomaterials.
   Ann N Y Acad Sci. 2001 November; 944: 187-98.
75: Matsumoto K, Nakamura T, Fukuda S, Sekine T, Ueda H, Shimizu Y.
   A gelatin coated collagen-polyglycolic acid composite membrane as a dural substitute.
   ASAIO J. 2001 November-December; 47(6):641-5.
76: Hartzerink J D, Beniash E, Stupp S I
   Self-assembly and mineralization of peptide-amphiphile nanofibers.
   Science. 2001 Nov. 23; 294(5547):1684-8.
78: Xu B, Gu Y, Mivamoto M, Balamurugan A N, Cui W, Imamura M, Iwata H, Inoue K,
   The influence of the anticomplement synthetic sulfonic polymers on the function of pancreatic islets: an in vitro study.
   Cell Transplant. 2001; 10(4-5):413-7.
79: Prokop A, Kozlov E, Nun Non S, Dikov M M, Sephel G C, Whitsitt J S, Davidson J M
   Towards retrievable vascularized bioartificial pancreas: induction and long-lasting stability of polymeric mesh implant vascularized with the help of acidic and basic fibroblast growth factors and hydrogel coating.
   Diabetes Technol Ther. 2001 Summer; 3(2):245-61.
80: Nakamura T, Ueda H, Tsuda T, Li Y H, Kivotani T, Inoue M, Matsumoto K, Sekine T, Yu L, Hvon S H, Shimizu Y.
   Long-term implantation test and tumorigenicity of polyvinyl alcohol hydrogel plates.
   J Biomed Mater Res. 2001 August; 56(2):289-96.
81: Alberti C.
   From the intestinal neobladder to the bioartificial bladder: remarks on some biological implications.
   Minerva Urol Nefrol. 2000 December; 52(4):219-22.
82: Ohsumi T K, Flaherty J E, Barocas V H, Adjerid S, Aiffa M.
   Adaptive Finite Element Analysis of the Anisotropic Biphasic Theory of Tissue-Equivalent Mechanics.
   Comput Methods Biomech Biomed Engin. 2000; 3(3):215-229.
83: Mullen Y, Maruvama M, Smith C V.
   Current progress and perspectives in immunoisolated islet transplantation.
   J Hepatobiliary Pancreat Surg. 2000; 7(4):347-57. Review.
84: Sakai S, Ono T, Ijima H, Kawakami K.
   Control of molecular weight cut-off for immunoisolation by multilayering glycol chitosan-alginate polyion complex on alginate-based microcapsules.
   J Microencapsul. 2000 November-December; 17(6):691-9.
   PMID: 11063416 [PubMed—indexed for MEDLINE]
85: Shi Q, Mitteregger R, Falkenhagen D, Yu Y T.
   A novel configuration of bioartificial liver support system based on circulating microcarrier culture.
   Artif Cells Blood Substit Immobil Biotechnol. 2000 July; 28(4):273-91.
86: Mizuguchi T, Mitaka T, Sato F, Mochizuki Y, Hirata K.
   Paper is a compatible bed for rat hepatocytes.
   Artif Organs. 2000 April; 24(4):271-7.
98: Pariente J L, Bordenave L, Bareille R, Baquev C, Le Guilt u M.
   Cultured differentiated human urothelial cells in the biomaterials field.
   Biomaterials. 2000 April; 21(8):835-9. Review.
   PMID: 10721752 [PubMed—indexed for MEDLINE]
99: Mayer J, Karamuk E, Akaike T, Wintermantel E.
   Matrices for tissue engineering-scaffold structure for a bioartificial liver support system.
   J Control Release. 2000 Feb. 14; 64(1-3):81-90.
100: Yamamoto Y, Nakamura T, Shimizu Y, Takimoto Y, Matsumoto K, Kivotani T, Yu L, Ueda H Sekine T, Tamura N.
   Experimental replacement of the thoracic esophagus with a bioabsorbable collagen sponge scaffold supported by a silicone stent in dogs.
   ASA10 J. 1999 July-August; 45(4):311-6.
101: J Biomater Sci Polym Ed 1998; 9(7):731-48
   Skeletal myogenesis on elastomeric substrates: implications for tissue engineering. Mulder M M, Hitchcock R W, Tresco P A.
   University of Utah, Department of Bioengineering, Salt Lake City 84112, USA. Studies geared towards understanding the interaction between skeletal muscle and biomaterials may provide useful information for the development of various emerging technologies, ranging from novel delivery vehicles for genetically modified cells to fully functional skeletal muscle tissue. To determine the utility of elastomeric materials as substrates for such applications, we asked whether skeletal myogenesis would be supported on a commercially available polyurethane, Tecoflex SG-80A. G8 skeletal myoblasts were cultured on Tecoflex two-dimensional solid thin films fabricated by a spin-casting method. Myoblasts attached, proliferated, displayed migratory activity and differentiated into multinucleated myotubes which expressed myosin heavy chain on solid thin films indicating that Tecoflex SG-80A was permissive for skeletal myogenesis. Porous three-dimensional (3-D) cell scaffolds were fabricated in a variety of shapes, thicknesses, and porosities by an immersion precipitation method, and where subsequently characterized with microscopic and mechanical methods. Mechanical analysis revealed that the constructs were elastomeric, recovering their original length following 100% elongation. The 3-D substrates were seeded with muscle precursors to determine if muscle differentiation could be obtained within the porous network of the fabricated constructs. Following several weeks in culture, histological studies revealed the presence of multinucleated myotubes within the elastomeric material. In addition, immunohistochemical analysis indicated that the myotubes expressed the myosin heavy chain protein suggesting that the myotubes had reached a state of terminal differentiation. Together the results of the study suggest that it is indeed feasible to engineer bioartificial systems consisting of skeletal muscle cultivated on a 3-D elastomeric substrate.

### GENETIC TRANSFER/MUTATION REFERENCES

102: Gournay J, Auvigne I, Pichard V, Ligeza C, Bralet M P, Ferry N.
   In vivo cell lineage analysis during chemical hepatocarcinogenesis in rats using retroviral-mediated gene transfer: evidence for dedifferentiation of mature hepatocytes.
   Lab Invest. 2002 June; 82(6):781-8.
103: Tawadros T, Meda P, Leisinger H J, Waeber G, Haefliger J A.
   Connexin26 is regulated in rat urothelium by the scaffold protein IB1/JIP-1.
   Cell Adhes Commun. 2001; 8(4-6):303-6.
104: Jones J M, Wilson K H, Koch W J, Milano C A.
   Adenoviral gene transfer to the heart during cardiopulmonary bypass: effect of myocardial protection technique on transgene expression.
   Eur J Cardiothorac Surg. 2002 May; 21(5):847-52. No abstract available.
105: Thanh H N, Murakami A, Fujiki K, Kanai A.
   Transferrin-Polyethylenimine Conjugate, FuGENE6 and TransIT-LT as Nonviral Vectors for Gene Transfer to the Corneal Endothelium.
   Jpn J Ophthalmol. 2002 March-April; 46(2):140-6. P
106: Varady P, Li J Z, Alden T D, Kallmes D F, Williams M B, Helm G A. CT and radionuclide study of BUT-2 gene therapy-induced bone formation. Acad Radiol. 2002 June; 9(6):632-7.
107: Josephson N C, Vassilopoulos G, Trobridge G D, Priestley G V, Wood B L, Papayannopoulou T, Russell D W.
   Transduction of human NOD/SCID-repopulating cells with both lymphoid and myeloid potential by foamy virus vectors.
   Proc Natl Acad Sci U S A. 2002 Jun. 11; 99(12):8295-300.
108: Oakley R, Phillips E, Hooper R, Wilson D, Partridge M.
   A Preclinical Model of Minimal Residual Cancer in the Muscle Highlights Challenges Associated with Adenovirus-mediated p53 Gene Transfer.
   Clin Cancer Res. 2002 June; 8(6):1984-94.
109: Bernal R M, Sharma S, Gardner B K, Douglas J T, Bergelson J M, Dubinett S M, Batra R K.
   Soluble coxsackievirus adenovirus receptor is a putative inhibitor of adenoviral gene transfer in the tumor milieu.
   Clin Cancer Res. 2002 June; 8(6):1915-23.
110: Mercapide J, Lopez De Cicco R, Bassi D E, Castresana J S, Thomas G, Klein-Szanto A J. Inhibition of Furin-mediated Processing Results in Suppression of Astrocytoma Cell Growth and Invasiveness.
   Clin Cancer Res. 2002 June; 8(6):1740-6.
111: Emtage P C, Xing Z, Wan Y, Zlotnik A, Graham F L, Gauldie J.
   Adenoviral-Mediated Gene Transfer of Lymphotactin to the Lungs of Mice and Rats Results in Infiltration and Direct Accumulation of CD4+, CD8+, and NK Cells.
   J Interferon Cytokine Res. 2002 May; 22(5):573-82.
112: Ogawa H, Kobayashi T, Yokoyama I, Nagatani N, Mizuno M, Yoshida J, Kadomatsu K, Muramatsu H, Nakao A, Muramatsu T.
   Reduction of alpha-galactosyl xenoantigen by expression of endo-beta-galactosidase C in pig endothelial cells.
   Xenotransplantation. 2002 July; 9(4):290-6.
113: Radchuk V V, Ryschka U, Schumann G, Klocke E.
   Genetic transformation of cauliflower (Brassica oleracea var. botrytis) by direct DNA uptake into mesophyll protoplasts.
   Physiol Plant. 2002 March; 114(3):429-438
114: Ohmiya N, Emi N, Niwa Y, Goto H, Hayakawa T.
   Insulin-enhanced liposome-mediated gene transfer into a gastric carcinoma cell line.
   Clin Exp Pharmacol Physiol. 2002 July; 29(7):544-8.
115: Walther W, Stein U, Fichtner 1, Voss C, Schmidt T, Schleef M, Nellessen T, Schlag P M. Intratumoral low-volume jet-injection for efficient nonviral gene transfer.
   Mol Biotechnol. 2002 June; 21(2):105-15.
117: Margetts P J, Kolb M, Yu L, Hoff C M, Holmes C J, Anthony D C, Gauldie J.
   Inflammatory cytokines, angiogenesis, and fibrosis in the rat peritoneum.
   Am J Pathol. 2002 June; 160(6):2285-94.
118: Bednarek I, Czajka M, Wilczok T.
   Efficiency of lipofection of adherent cells is limited by apoptosis.
   Folia Histochem Cytobiol. 2002; 40(2):133-4.
120: Yang Z, Chen M, Wu R, Fialkow L B, Bromberg J S, McDuffie M, Naji A, Nadler J L.
   Suppression of autoimmune diabetes by viral IL-10 gene transfer.
   J Immunol. 2002 Jun. 15; 168(12):6479-85.
123: Sakamoto M, Yuasa K, Yoshimura M, Yokota T, Ikemoto T, Suzuki M, Dickson G, MiyagoeSuzuki Y, Takeda S.
   Micro-dystrophin cDNA ameliorates dystrophic phenotypes when introduced into mdx mice as a transgene. Biochem Biophys Res Commun. 2002 May 17; 293(4):1265-72.
126: Sylven C, Sarkar N, Insulander P, Kenneback G, Blomberg P, Islam K, Drvota V.
   Catheter-based transendocardial myocardial gene transfer.
   J Interv Cardiol. 2002 Febuary; 15(1):7-13.
128: Kanzaki S, Kawamoto K, Oh S H, Stover T, Suzuki M, Ishimoto S, Yagi M, Miller J M, Lomax M I, Raphael Y.
   From gene identification to gene therapy.
   Audiol Neurootol. 2002 May-June; 7(3):161-4.
129: Holt J R.
   Viral-mediated gene transfer to study the molecular physiology of the Mammalian inner ear.
   Audiol Neurootol. 2002 May-June; 7(3):157-60.
130: Lalwani A K, Jero J, Mhatre A N.
   Current issues in cochlear gene transfer.
   Audiol Neurootol. 2002 May-June; 7(3):146-51.
134: Davis H E, Morgan J R, Yarmush M L.
   Polybrene increases retrovirus gene transfer efficiency by enhancing receptor-independent virus adsorption on target cell membranes.
   Biophys Chem. 2002 Jun. 19; 97(2-3):159-72.
135: Copley S D, Dhillon J K.
   Lateral gene transfer and parallel evolution in the history of glutathione biosynthesis genes. Genome Biol. 2002; 3(5):RESEARCH0025.
136: Yanai I, Wolf Y I, Koonin E V.
   Evolution of gene fusions: horizontal transfer versus independent events. Genome Biol. 2002; 3(5):RESEARCH0024.
137: Villani G R, Follenzi A, Vanacore B, Di Domenico C, Naldini L, Di Natale P.
   Correction of mucopolysaccharidosis type I1Ib fibroblasts by lentiviral vector-mediated gene transfer.
   Biochem J. 2002 Jun. 15; 364(Pt 3):747-53.
138: Bonatti J, Haeusler C, Klaus A, Fink M, Hammerer-Lercher A, Laufer G.
   Acceptance of gene therapy by the heart surgery patient.
   Eur J Cardiothorac Surg. 2002 June; 21(6):981-6.
139: Syvanen M.
   Recent emergence of the modem genetic code: a proposal.
   Trends Genet. 2002 May; 18(5):245-8.
140: Di Natale P, Di Domenico C, Villani G R, Lombardo A, Follenzi A, Naldini L. 1 In vitro gene therapy of mucopolysaccharidosis type I by lentiviral vectors.
   Eur J Biochem. 2002 June; 269(11):2764-71.
141: Thanh H N, Murakami A, Fujiki K, Kanai A. Related Articles Transferrin-Polyethylenimine Conjugate, FuGENE6 and TransIT-LT as Nonviral Vectors for Gene Transfer to the Comeal Endothelium.
   Jpn J Ophthalmol. 2002 March-April; 46(2):140-6.
142: Walther W, Stein U, Fichtner I, Voss C, Schmidt T, Schleef M, Nellessen T, Schlag P M. Related Articles
   Intratumoral low-volume jet-injection for efficient nonviral gene transfer. Mol Biotechnol. 2002 June; 21(2):105-15.
143: Sylven C, Sarkar N, Insulander P, Kenneback G, Blomberg P, Islam K, Drvota V. Related Articles
   Catheter-based transendocardial myocardial gene transfer. J Interv Cardiol. 2002 Febuary; 15(1):7-13.
145: Filenko N A, Shklyaruk V V, Butenko G M, Kavsan V M.
   Plasmid mediated expression of human growth hormone increases splenocytes proliferative response in vivo.
   Ukr Biokhim Zh. 2001 May-June; 73(3):30-3.
146: Reed C C, Gauldie J, lozzo R V. Related Articles
   Suppression of tumorigenicity by adenovirus-mediated gene transfer of decorin. Oncogene. 2002 May 23; 21(23):3688-95.
147: Fajac 1, Grosse S, Briand P, Monsigny M. Related Articles
   Targeting of cell receptors and gene transfer efficiency: a balancing act. Gene Ther. 2002 June; 9(11):740-2.
148: Delepine P, Montier T, Guillaume C, Vaysse L, Le Pape A, Ferec C. Related Articles
   Visualization of the transgene distribution according to the administration route allows prediction of the transfection efficacy and validation of the results obtained.
   Gene Ther. 2002 June; 9(11):736-9.
149: Schindelhauer D, Laner A. Related Articles
   Visible transient expression of EGFP requires intranuclear injection of large copy numbers.
   Gene Ther. 2002 June; 9(11):727-30.
150: Hanenberg H, Batish S D, Pollok K E, Vieten L, Verlander P C, Leurs C, Cooper R J, Gottsche K, Haneline L, Clapp D W, Lobitz S, Williams D A, Auerbach A D. Related Articles
   Phenotypic correction of primary Fanconi anemia T cells with retroviral vectors as a diagnostic tool. Exp Hematol. 2002 May; 30(5):410-20.
152: Liu F, Huang L. Related Articles
   Noninvasive gene delivery to the liver by mechanical massage. Hepatology. 2002 June; 35(6):1314-9.
153: Filee J, Forterre P, Sen-Lin T, Laurent J. Related Articles
   Evolution of DNA polymerase families: evidences for multiple gene exchange between cellular and viral proteins.
   J Mol Evol. 2002 June; 54(6):763-73.
154: Masaki I, Yonemitsu Y, Yamashita A, Sata S, Tanii M, Komori K, Nakagawa K, Hou X, Nagai Y, Hasegawa M, Sugimachi K, Sueishi K. Related Articles
   Angiogenic gene therapy for experimental critical limb ischemia: acceleration of limb loss by overexpression of vascular endothelial growth factor 165 but not of fibroblast growth factor-2. Circ Res. 2002 May 17; 90(9):966-73.
155: Segura T, Shea L D. Related Articles Surface-tethered DNA complexes for enhanced gene delivery. Bioconjug Chem. 2002 May-June; 13(3):621-9.
156: Kreppel F, Luther T T, Semkova I, Schraermeyer U, Kochanek S.
   Long-Term Transgene Expression in the RPE after Gene Transfer with a High-Capacity Adenoviral Vector.
   Invest Ophthalmol Vis Sci. 2002 June; 43(6):1965-70.
157: Friedman J M, Horwitz M S. Related Articles
   Inhibition of tumor necrosis factor alpha-induced NF-kappa B activation by the adenovirus E3-10.4/14.5K complex.
   J Virol. 2002 June; 76(11):5515-21.
158: Li Y, McCadden J, Ferrer F, Kruszewski M, Carducci M, Simons J, Rodriguez R. Related Articles
   Prostate-specific expression of the diphtheria toxin A chain (DT-A): studies of inducibility and specificity of expression of prostate-specific antigen promoter-driven DT-A adenoviral-mediated gene transfer. Cancer Res. 2002 May 1; 62(9):2576-82.
159: Heinkelein M, Dressler M, Jarmy G, Rammling M, Imrich H, Thurow J, Lindemann D, Rethwilm A. Related Articles
   Improved primate foamy virus vectors and packaging constructs. J Virol. 2002 April; 76(8):3774-83.
160: Kost T A, Condreay J P. Related Articles
   Recombinant baculoviruses as mammalian cell gene-delivery vectors. Trends Biotechnol. 2002 April; 20(4):173-80. Review.
162: Kapturczak M H, Flotte T, Atkinson M A. Related Articles
   Adeno-associated virus (AAV) as a vehicle for therapeutic gene delivery: improvements in vector design and viral production enhance potential to prolong graft survival in pancreatic islet cell transplantation for the reversal of type 1 diabetes.
   Cuff Mol Med. 2001 May; 1(2):245-58.
163: Takebe N, Xu L C, MacKenzie K L, Bertino J R, Moore M A. Related Articles
   Methotrexate selection of long-term culture initiating cells following transduction of CD34(+) cells with a retrovirus containing a mutated human dihydrofolate reductase gene.
   Cancer Gene Ther. 2002 March; 9(3):308-20.
164: Tunnacliffe A, Parkar M, Povey S, Bengtsson B O, Stanley K, Solomon E, Goodfellow P. Related Articles
   Integration of Ecogpt and SV40 early region sequences into human chromosome 17: a dominant selection system in whole cell and microcell human-mouse hybrids. EMBO J. 1983; 2(9):1577-84.
165: Matthias P D, Bernard H U, Scott A, Brady G, Hashimoto-Gotoh T, Schutz G. Related Articles
   A bovine papilloma virus vector with a dominant resistance marker replicates extrachromosomally in mouse and E. coli cells.
   EMBO J. 1983; 2(9):1487-92.
166: Negre D, Duisit G, Mangeot P E, Moullier P, Darlix J L, Cosset F L. Related Articles
   Lentiviral vectors derived from simian immunodeficiency virus.
   Curr Top Microbiol Immunol. 2002; 261:53-74. Review. No abstract available.
167: Stevenson M. Related Articles
   Molecular biology of lentivirus-mediated gene transfer.
   Curr Top Microbiol Immunol. 2002; 261:1-30. Review. No abstract available.
168: Peng X, Blum H, She Q, Mallok S, Brugger K, Garrett R A, Zillig W, Prangishvili D. Related Articles, Nucleotide, Protein
   Sequences and replication of genomes of the archaeal rudiviruses SIRVI and SIRV2: relationships to the archaeal lipothrixvirus SIFV and some eukaryal viruses.
   Virology. 2001 Dec. 20; 291(2):226-34.
169: Stitz J, Muhlebach M D, Blomer U, Scherr M, Selbert M, Wehner P, Steidl S, Schmitt I, Konig R, Schweizer M, Cichutek K. Related Articles
   A novel lentivirus vector derived from apathogenic simian immunodeficiency virus. Virology. 2001 Dec. 20; 291(2):191-7.
170: Sapru M K, McCormick K M, Thimmapaya B. Related Articles
   High-efficiency adenovirus-mediated in vivo gene transfer into neonatal and adult rodent skeletal muscle. JNeurosci Methods. 2002 Feb. 15; 114(1):99-106.

What is claimed is:

1. An artificial connective tissue, comprising:
a plurality of elongated fibers each having a first end and a second end;
a first end fiber at said first end of said elongated fibers; and
a second end fiber at said second end of said elongated fibers,
wherein said first and said second end fibers connect said respective first and second ends of said elongated fibers.

2. An artificial connective tissue according to claim 1, further comprising at least one cross-connecting fiber attached to at least two of said elongated fibers between said first ends and said second ends.

3. An artificial connective tissue according to claim 1, further comprising a layer of fibroblast or tendon cells on said elongated fibers.

4. An artificial weight-bearing bone, comprising:
a plurality of elongated members;
a plurality of circular fibers; and
at least one layer of bone or cartilage cells on at least one of said elongated members,
wherein said elongated members form a generally cylindrical structure,
wherein said circular fibers are located around and permanently attached to said elongated members, and
wherein said circular fibers are spaced along the length of said elongated members.

5. An artificial weight-bearing bone according to claim 4, wherein said elongated members are comprised of metal.

6. An artificial weight-bearing bone according to claim 4, wherein the artificial weight-bearing bone is selected from the group consisting of: artificial femur, artificial tibia, artificial fibula, artificial rib, artificial clavicle, artificial humerus, artificial radius and artificial ulna.

7. An artificial weight-bearing bone according to claim 4, wherein the artificial weight-bearing bone mimics the shape of a naturally occurring bone or portion thereof.

8. An artificial weight-bearing bone, comprising:
a plurality of elongated members;
a plurality of circular fibers; and
at least one layer of bone or cartilage cells on at least one of said elongated members,
wherein said elongated members form a structure that mimics the shape of a natural bone or portion thereof, and
wherein each of said circular fibers are located around and permanently attached to at least two of said elongated members.

9. An artificial weight-bearing bone according to claim 8, wherein said elongated members are comprised of metal.

10. An artificial weight-bearing bone according to claim 8, wherein the artificial weight-bearing bone is selected from the group consisting of: artificial scapula, artificial vertebra, artificial inferior maxillary, artificial sternum, artificial patella and artificial os innominatum.

11. An artificial non weight-bearing bone, comprising:
at least one artificial bone scaffold; and
at least one layer of bone or cartilage cells on said artificial bone scaffold,
wherein said artificial bone scaffold is configured to mimic the shape of a natural bone or a portion thereof.

12. An artificial non weight-bearing bone according to claim 11, further comprising a layer of cell growth matrix located along the inner or outer surface of said artificial bone scaffold.

13. An artificial non weight-bearing bone according to claim 11, wherein said artificial non weight-bearing bone is selected from the group consisting of: artificial nose and artificial ear.

14. An artificial skin panel, comprising:
a fibrous matrix; and
at least one cellular coating on said fibrous matrix,
wherein said at least one cellular coating is comprised of cells selected from the group consisting of superficial cutaneous cells and deep cutaneous cells.

15. An artificial cell growth cage, comprising:
a fibrous matrix; and
a plurality of cells on and within said fibrous matrix,
wherein said cells are capable of producing a desired gene expression product.

16. An artificial cell growth cage according to claim 15, wherein the artificial cell growth cage is tube shaped.

17. An artificial cell growth cage according to claim 16, wherein the artificial cell growth cage has an outer diameter of 10-16 gauge and an inner diameter of 0.5-0.05 mm.

18. An artificial cell growth cage according to claim 15, wherein the artificial cell growth cage is disk shaped.

19. An artificial cell growth cage according to claim 18, wherein the artificial cell growth cage has an outer diameter of 1-3 cm, a length of 3-6 cm and a width of 25-100 mm.

20. A cellular growth chamber, comprising:
a vessel;
an opening in said vessel that allows insertion and removal of a plurality of porous inner tubes and that is sealingly closeable; and
a sealable port providing a opening in said vessel and allowing inlet and outlet of cell culture solution.

21. A method of obtaining a substance, comprising:
culturing cells in a cellular growth chamber according to claim 20; and
isolating a substance produced by said cells.

22. An isolated substance obtained according to claim 21, wherein the substance is selected from the group consisting of: myostatin and follistatin.
